(19) United States
(12) Patent Application Publication
(10) Pub. No.: US 2023/0420108 A1
(43) Pub. Date: Dec. 28, 2023

(54) SYSTEM FOR COMPOSING INDIVIDUALIZED NUTRITION FOR OR FROM DIFFERENT RESOURCES

(71) Applicant: LOEWI GmbH, Berlin (DE)

(72) Inventor: Philipp Merk, Garching (DE)

(21) Appl. No.: 18/037,284

(22) PCT Filed: Nov. 9, 2021
(86) PCT No.: PCT/EP2021/081118

§ 371 Date: May 16, 2023

(30) Foreign Application Priority Data
Nov. 18, 2020 (EP) 20208436.4

(51) Int. Cl. G16H 20/60; A23L 33/10; A23L 33/00

(52) U.S. Cl. CPC G16H 20/60; A23L 33/10; A23L 33/30

(57) ABSTRACT

Disclosed is a system for automatically composing individualized nutrient compositions from a plurality of nutrient(s) comprising products. The system comprises a processing module configured to receive entity data, receive product nutrients data and to individually compose nutrients on the basis of the entity data and the product nutrients data. The system further comprises a node which is configured to receive entity data in form of a user input and provide the entity data to the processing module.

### FIELD

The present invention relates to a system and a method to automatically compose an individualized nutrition al composition for or from different resources.

### BACKGROUND

A person's body cannot produce everything that it needs to function. There are essential nutrients that people need to consume through dietary sources to maintain optimal health. The World Health Organization (WHO) note that essential nutrients are crucial in supporting a person's reproduction, good health, and growth. The WHO divide these essential nutrients into two categories: micronutrients and macronutrients. Micronutrients are nutrients that a person needs in small doses. Micronutrients consist of vitamins and minerals. Although the body only needs small amounts of them, a deficiency can cause ill health. Macronutrients are nutrients that a person needs in larger amounts. Macronutrients include water, protein, carbohydrates, and fats. The six essential nutrients are vitamins, minerals, protein, fats, water, and carbohydrates.

Vitamins are micronutrients that offer a range of health benefits, including: boosting the immune system, helping prevent or delay certain cancers, strengthening teeth and bones, aiding calcium absorption, maintaining healthy skin, helping the body metabolize proteins and carbs, supporting healthy blood, aiding brain and nervous system functioning etc.

There are a number of essential vitamins that nutritionists divide into two groups: fat soluble and water soluble. Fat soluble vitamins are: vitamins A, D, E, K. Water soluble vitamins are: vitamins B-1 (thiamine), B-12 (cyanocobalamin), B-6, B-2 (riboflavin), vitamin B-5 (pantothenic acid), vitamin B-3 (niacin), vitamin B-9 (folate, folic acid), vitamin B-7 (biotin), vitamin C.

Ideally, a person who eats a diet rich in vegetables, fruits, and lean proteins can get all the vitamins they need in their food. However, the vast majority of people have no access to or eat less fruit and vegetables, and those with digestive conditions may need to take a vitamin supplement to reduce or avoid a deficiency.

Minerals are the second type of micronutrients. There are two groups of minerals: major and trace minerals. The body needs a balance of minerals from both groups for optimal health.

Major minerals are magnesium, calcium, phosphorus, sulfur, sodium, potassium, chloride. Major minerals help the body to do the following: balance water levels, maintain healthy skin, hair, and nails and improve bone health.

Trace minerals are: iron, selenium, zinc, manganese, chromium, copper, iodine, fluoride, molybdenum. Trace minerals help with strengthening bones, preventing tooth decay, aiding in preventing blood clotting, helping to carry oxygen, supporting the immune system, supporting healthy blood pressure.

This already shows that the appropriate consumption of vitamins and minerals is a difficult if not impossible task. It is getting even more complicated as depending on lifestyle, genetic predisposition, epigenetic impact, climate, seasons etc. the needs can vary very substantially.

Thus, strong attempts have been made to assist people to properly consume nutrients.

For example, EP 3469359 A1 discloses a method for providing nutritional supplement information for a subject (1) is proposed, said method including a sequence of the following steps in given order: A) taking a sample (3) from the subject (1); B) analysing said sample to determine the nutritional status of the subject (1); C) based on the results calculation of the required nutritional supplements to improve the nutritional status of the subject (1); D) providing individualized nutritional supplement information to the subject (1). This sequence of A)-D) is repeated at least once after a time span of at least 2 days or one week for adapting the provided nutritional information in step D) of the following sequence based on the development of the subject (1). Step C) involves the prediction of at least one initial characteristics matrix and the multiplication of this matrix weighted with weighting factors, with an initial recommendation vector for the calculation of a target profile vector after a given first time interval from the profile vector as determined in step B), and in each following cycle adaptation in that the difference between the previously calculated target profile vector and the profile vector as determined in the actual analysis step B) is minimized by adapting at least one of the characteristics matrix and the weighting factors and using the adapted for the calculation of the required next recommendation vector.

U.S. Pat. No. 7,983,932 B2 provides networks and a method for linking consumers and nutritional pharmacologists offering personalized nutritional information through a central network site. The network includes a central integration site through which network members communicate with each other. The central integration site stores two or more data bases in the storage medium. The databases store biochemical marker data information, nutritional and/or drug data information including a record for association and effect of nutrients with a particular biochemical marker, and/or drug. The network of the invention provides individualized nutritional diagnostic and treatment to consumers on the basis of their clinical test results.

U.S. Pat. No. 8,762,167 B2 provides method and systems for generation of personalized health plans. Personalized, health and performance programs are generated for individuals based on various biomarkers and performance and lifestyle assessments. In one embodiment, a diagnostic test of blood or other biological specimen(s) is used to determine key biological marker levels. Information and assessments of the user's physical performance, lifestyle and health and wellness goals are also collected and provided to an expert system that matches the biomarker levels and assessments to a knowledgebase of scientific knowledge about biomarker levels and health and fitness outcomes. Personal recommendations and advice on nutrition and exercise is then generated, which may be used to help individuals reach their diet, fitness, and wellness goals and improve their physical and mental performance and well-being in measurable ways.

However, nutrients are included in countless kinds and brands of food, drinks, functional food, specific nutrients by sometimes even one supplier let alone the number of respective products by a number or all suppliers. Thus, there is still no support to reliably individualize nutrients of one or more suppliers for a person. Moreover, there is no picture what nutrients a specific person has in fact consumed already, what is still needed and how the needed nutrients could be delivered. The detailed information of nutrients contained are also not standardized and cannot often be compared.

### SUMMARY

In light of the above, it is an object of the present invention to overcome or at least alleviate the shortcomings of the prior art. More particularly, it is an object of the present invention to provide automatically compounded and individualized nutrient compositions automatically individualize nutrients, respectively to automatize providing individualized nutrient compositions. These objects are fulfilled by the method and system of the present invention.

The present invention relates to a system and method for automatically composing individualized nutrient compositions from different nutrient(s) comprising products. It can compose or recommend the doses of just one nutrient or the composition and/or doses and/or medium of nutrients.

The system can be configured to automatically compose an individualized nutrition, respectively nutrient composition, based on a plurality of nutritionally relevant data provided to the system. Furthermore, the system can be configured to provide individualized nutrition data to a data processing entity.

The system can comprise the processing or a processing module (1) configured to receive entity data and nutrient(s) data and to individually compose nutrients on the basis of the entity data.

The entity data comprises data specific to the entity the data relates to. The entity can is most broadly defined as a living organism, most commonly a human, animal or plant.

Furthermore, the entity can be a group of people, a race of animal or type of plant, wherein the entity data contains at least one data which is identical with respect to all individuals of the grouped organisms. For example, people can share a common blood marker, are of the same age, match in dietary intake, share a certain allergy, etc.

The entity data can comprise biometric data, medical data, i.e. DNA-marker data, blood count data, blood chemistry data, enzyme specific data, risk factors, dietary information, sensitivity to specific allergens. More generally, any body-, respectively health-quantifying data can be included.

Additionally, the entity data can comprise supplier data, wherein the supplier data can comprise a specific set of suppliers and/or manufacturers, specifics about where nutritional products need to be available and/or need to be shipped to.

The nutrient data, can comprise product data, which is specific to an existing nutrient product. Furthermore, the nutrient data can comprise data on at least on specific nutrient. With regard to the respective nutrient, the nutrient data can, for example, comprise dosage information, recommended daily intake values, nutrient cross link information specifying interactions with other nutrients, associated risks and/or benefits, combinatorial data linking the nutrient to other nutrients, typical dosage and/or delivery form, recommended combinatorial dietary limitations associated with the specific nutrient.

The term “individually” relates to the processing of the provided entity data. The processing module can be configured to process the provided entity data and generate a nutrient composition which is specific to that entity.

The data can be provided by the entity and/or by a third party via a node, in particular a network node, which can be a user interface node.

The term “composing” can comprise generating a dataset comprising nutrient product data. The nutrient product data can comprise data relating to the type of product, supplier(s) of the product, manufacturer of the product, nutritional content of the product and/or a recommendation of the product, in particular, a recommendation based on the provided entity data. The dataset can comprise multiple entries wherein each entry relates to a specific nutrient product by one or more suppliers, the combination of nutrient products by one or more suppliers and/or the aggregating of one or more products.

The composing can comprise the doses, the chemical and/or physical form of the nutrient(s) and their composition to each other.

The term “nutrient composition” comprises a composition of nutrients by means of one or more products comprising nutrients. Each product can comprise a plurality of nutrients or a single nutrient. Furthermore, a specific nutrient can be present in different products of a nutrient composition. In particular, the concentration of the nutrient can vary by product.

A product can be specified by the amount of a specific nutrient per weight or volume of the product substance, i.e. the nutrient concentration. The product can be a compound of nutrients, a combination of solvent and nutrients a mixture of nutrients or any combination of the aforementioned.

the products can comprise nutrient compositions, wherein a specific nutrient can be part of different products

It is a preferred advantage of the present invention to easily and reliably individualize or personalize nutrients and nutrient composition reliably. Moreover, the individual can also be heard and has the feeling of an active contribution.

The composing can further aid in preventing overdosing, triggering of allergies, aid in limiting chemical interactions of nutrients or any other non-desirable effects.

A nutrient composition can fulfil or supplement nutrient requirements of an entity. According to an embodiment of the invention the nutrient composition can be aimed at increasing physiological and/or neurological functions of the entity. In particular, the nutrient composition can include nutrients which may benefit tissue regeneration, optimize nutrient balance on an overall and/or cellular level, benefit transmission between neurons and/or overall increase neural efficiency.

Further, it is a preferred advantage of the present invention to quantify the nutrient intake of an entity, respectively individual, to prevent or alleviate nutritional deficits and/or nutrient deficiencies.

According to a first aspect the invention relates to a system for automatically composing individualized nutrient compositions from a plurality of nutrient(s) comprising products.

The system comprises a processing module configured to receive entity data and to individually compose nutrients on the basis of the entity data. This achieves the advantage that a nutrient composition can be generated automatically and in particular systematically. The processing module is configured to deterministically arrive at a nutrient composition based on the available entity data. The nutrient composition can be matched to an individual represented by the entity data and can thus be specifically tailored to the nutritional requirements of that individual.

The individualized nutrient composition can comprise only one nutrient product. The only one nutrient product may contain a single nutrient as an active ingredient or contain a plurality of nutrients as active ingredients. The minimal configuration of the plurality of nutrient(s) comprising products comprises two nutrient products, wherein each of the two nutrient products contains at least one nutrient as an active ingredient. The nutrients can be identical and the nutrient products can be differentiated by nutrient product properties, i.e. dosage, delivery form, volume, number of single doses etc.

The processing module can receive and process product nutrient(s) data and/or generate a nutrient composition based on the entity data and nutrient(s) data. This achieves the advantage that the nutrient composition can be based on existing combinations of nutrients, respectively nutrient products which contain the required nutrient according to the generated nutrient composition in a desired form (i.e. dosage, concentration, bulk medium).

A node can provide the entity data. In particular the node can be understood as a user input or a user input terminal. The node can be an entity-controlled device which enables an entity, respectively an individual to wilfully submit entity into the system. The data can be submitted in any form. The entity data can comprise a consent information by the entity to submit data to the system.

The node can receive the entity data in form of a user input. In particular the node can comprise a human interface device (HID) configured to directly interact with an individual to allow the entity to input entity data to the node. The HID can comprise a tactile, voice-enabled, video-enabled.

According to an embodiment the node can communicate with the processing module, and is thereby configured to transmit entity data to the processing module and/or to receive a nutrient composition from the processing module. The node can comprise an output module configured to transmit information on the nutrient composition to an entity present at the node. In particular the node can comprise a screen, speaker, printer and/or a Braille output device.

The node can authenticate an entity to the processing module and/or a node interface module. The authentication can comprise encryption, in particular symmetric or asymmetric encryption involving keys. Furthermore, the node can authenticate an entity to the processing module for receiving a nutrient composition. The authentication can be linked to a payment verification, wherein the nutrient composition is provided when a payment by the entity is registered by the node, by the processing module and/or by the node interface module.

The system can comprise a node interface module which can communicate with nodes to receive entity data from the nodes. The node interface module can act as a hub to a plurality of nodes. In that function the node interface module can aggregate entity data and send aggregated entity data to the processing module. The communication can be either initiated by the processing module or the node interface module.

Furthermore, the node interface module can receive local product nutrient(s) data from a local database module. The local product nutrient(s) data can comprise nutrient product data of nutrient products locally available at the node interface module. The node interface module can be located in a medical, pharmaceutical and/or any nutrient product(s) supplying facility which comprises a local stock of nutrient products. The nutrient composition composed by the processing module can be tailored to the locally available stock of nutrient products and/or other products. The local stock of nutrient products can be represented in a local database linked to the local database module. The local database can comprise a dataset on pharmaceutical products, nutrient products and/or food products. Locally available nutrient products can include nutrient products directly in stock at the facility and/or available to be shipped to the facility.

The node interface module can provide the local product nutrient(s) data to the processing module which is configured to compose a nutrient composition based on the local product nutrient(s) data. Thereby, the processing module can be limited to a subset of nutrient products available from the nutrient databases. Furthermore, the local product nutrient(s) data can have an overlap with the nutrient products listed in the product nutrient(s) database or form a separate set of nutrient products with no overlap in reference to the product nutrient(s) database.

The processing module and/or the node interface module can authenticate the entity, the user and/or the node by means of authentication provided by the node. The authentication data can initially be provided by the entity. The authentication means can be an identification document, ID card, insurance card or login credentials. The node can initiate a session with the node interface module and/or the processing module by providing login credentials as authentication means.

The processing module and/or the node interface module can receive the entity data when the node is authenticated. Thereby, transmitting and processing of entity data can be shielded from third party access. The node interface module can be integrated with the processing module.

The node interface module can trigger receipt and/or transmission of the entity data by the processing module and/or the node interface module. Thereby entity data provided to the node is not transmitted automatically to the processing module and/or the node interface module, but the user of the node is able to consent to the transmission of the input entity data. Furthermore, receiving of entity data from the processing module and/or the node interface module can be initiated by the node.

The node interface module can communicate with an external database module to receive entity data. This provides the advantage that additional entity data, in particular entity data not available from the node or not transmitted by the node, can be gathered and used as a basis for generating the nutrient composition.

Furthermore, the node can authenticate a communication of the node interface module with the external database module to initiate a transmission of entity data from the external database module to the node interface module. The transmitted entity data can be specific to the entity represented by the node which authenticates the communication. Thus, the entity can have full control over the specific entity data which is provided to the node interface module and/or the processing module. In particular, the entity can enforce limits regarding which entity data is available to the node interface module, respectively the processing module from the external database module.

The node interface module can authenticate the node and/or itself to the external database module to receive entity data specific to the entity represented by the node. The external database module may provide access to medical data regarding the entity. Thus, it is advantageous to limit access to the external database to authenticated entities, respectively an authenticated node and/or node interface module. The authentication can include a verification of the entity and authorization of access to a data transfer from the external database to the node interface module and/or the processing module. Therefore, the processing module can be configured to communicate with the external database module.

The node can authenticate a communication of the processing module with the external database module to initiate a transmission of entity data from the external database module to the processing module. The transmitted entity data is specific to the entity represented by the node which authenticates the communication. This achieves the advantage that the entity data from the external database can be directly sent to the processing module. In particular, the node can grant access to the processing module and/or the node interface module to gather additional entity data from the external database. Thus, the amount of entity data provided by the node can be reduced to data which is not yet present in the system, especially not present in the external database or an entity profile database linked to the processing module. The entity profile database can be a template database.

The processing unit can authenticate the node and/or itself to the external database module to receive entity data specific to the entity represented by the node. Thereby, achieving the advantage that the node does not need to provide authentication information. The node, respectively the entity providing entity data through the node authenticates itself to the processing module while the processing module manages the authentication to the external database module.

The external database module can be part of the system and can provide entity data to the processing module. Furthermore, the external database module can provide entity data to the node interface module and/or to the processing module. The entity data provided by the external database module can be specific to one individual, respectively one entity and pertains to the health condition, nutrient consumption, pharmaceutical intake, physical exercise and/or medical data of that individual. Furthermore, the entity data provided by the external database module can comprise nutritional-, exercise-, geolocation- and/or body function-tracking data. This data can be supplied by devices handled by, respectively linked to the entity. This achieves the advantage that the processing module is able to generate a nutrient composition based on detailed information concerning the health state of the individual, respectively entity. Thereby, greater accuracy of the provided nutrient composition.

The node can activate the entity interface module and vice versa. Additionally, the entity interface module can trigger communication with one or more of the nodes upon activation. In particular a communication session can be established between the node and the node interface module. In particular, the node can sequentially provide entity data to the node interface module, wherein the node interface module controls the type of entity data which can be provided in each communication sequence. For example, the entity provides a first entity datum and the node interface module can determine which type of entity data is additionally required and request that required entity data in a subsequent communication with the node. Thereby, a set of entity data can be generated at the node interface module which is tailored to the requirements of the processing module to generate the nutrient composition.

Additionally, the nodes can trigger communication with the entity interface module upon activation. Thus, a communication sequence or single transmission can be initiated by one of the nodes. Furthermore, the node interface module can communicate in dialogue with the entity node(s).

The system can comprise an entity profile database for storing entity data and the processing module can store entity data provided by the nodes in the entity profile database. Thus, recurring communication with a node does not require the submission of previously submitted entity data. However, when updated entity data of the same type of previously submitted entity data is present transmitting that data from the node to the processing module or the node interface module may be required.

The processing module can store entity data provided by the external database module in the entity profile database. Thereby, the advantage is achieved that a complete entity profile can be generated in the entity profile database decreasing the number of future data transfers required from the external database module or the node.

The processing module can merge first entity data provided by the nodes and second entity data provided by the external database module to generate merged entity data, wherein the processing module can compose nutrients on the basis of the merged entity data. This achieves the advantage that the processing module can derive a nutrient composition based on multiple variables. Moreover, the external database may provide chronological data of specific entity parameters. Thus, the processing module can take temporal entity data gradients into account when processing entity data and product nutrient(s) data to generate the nutrient composition. In particular, the nutrient composition can be tailored to counter entity data gradients, in particular gradients considered to be adverse to the health of the entity.

Furthermore, the processing module and/or the user interface module can anonymize received entity data and the entity profile database can store the anonymized entity data. Advantageously, the anonymized entity data can be used by the processing module to generate statistical data spanning multiple entity datasets while adhering to data privacy standards.

According to an advantageous embodiment the processing module can receive external entity data from the external database module and compose a nutrient composition based on the received external entity data. The nutrient composition may even be entirely based on external entity data. The node may control the access of the processing module to the external data specific to the entity represented by the node.

The processing module can filter and/or select product nutrient(s) data on which generating the nutrient composition is based. Filtering the product nutrient(s) data can be implemented by setting thresholds regarding specific nutrient properties. The processing module may generate these thresholds specific to the available entity data. The specific nutrient properties may include dosage information, recommended daily intake values, nutrient cross link information regarding nutrient interactions, typical dosage, bioavailability and/or delivery form.

The processing module can generate the filter based on the provided entity data to reduce a product interaction potential, a pharmaceutical interaction potential and/or an allergic reaction potential for the entity. Further, the processing module can apply that filter to the product nutrient(s) data to generate filtered product nutrient(s) data and/or to select a subset of the product nutrient(s) data. Then, the processing module can generate a nutrient composition on the basis of the filtered product nutrient(s) data.

A product interaction potential can, for example, include a factor regarding the likelihood of nutrient products, respectively their ingredients undergoing a reaction. The reaction can be a chemical reaction, where the chemical composition of the combined nutrient products changes and/or a mixing reaction, where the dosage form, respectively the physical form of the nutrient products changes, i.e. mixing a liquid and a powder, mixing polar and non-polar liquids.

A pharmaceutical interaction potential can quantify the likelihood of an adverse interaction of nutrients with pharmaceutical agents, i.e. an applicable nutrient dosage can be limited when the entity is medicated.

An allergic reaction potential can quantify the likelihood of the entity having an allergic reaction to the ingredients of nutrient products. This may concern inactive carrier ingredients and/or active nutrient ingredients of the nutrient products.

Selecting product nutrient(s) data can, for example, be implemented by choosing a subset from the product nutrient(s) data, in particular based on entity risk and benefits factors regarding the comprising nutrients of the nutrient products, combinatorial data linking the nutrient to other nutrients, typical dosage and/or delivery form, and/or recommended combinatorial dietary limitations associated with the specific nutrient, respectively nutrient product. Therefore, the processing module can also select a subset of product nutrient data to minimize a product interaction potential and/or a pharmaceutical interaction potential and/or an allergic reaction potential for the entity when the generated nutrient composition is consumed.

The system can further comprise the external database module configured to store entity data provided by an external source. The external source can be a medical database, hospital, any type of medical facility, a doctor, any type of health parameter tracking device database, a pharmacy, a nutrient product supplier database and/or a social media platform.

The entity profile database can contain a data profile which includes the entity data necessary to generate a nutrient composition for that entity. Furthermore, the data profile can comprise a chronological series of entity data enabling the processing module to generate a nutrient composition based on the chronological evolution of the entity data. The entity data stored in the entity profile database may represent a health profile, respectively a multidimensional health matrix. The multidimensional health matrix may link specific entity data elements and may define combinatorial thresholds. For example, a threshold for a first specific entity data element may be adjusted when a second specific entity data element exceeds its own threshold.

The node interface module can receive first entity data provided by the node, communicate with the external database module to receive second entity data, integrate the first entity and second entity data to generate combined entity data and to store the combined entity data in the entity profile database. Thus, a complete entity profile can be generated and stored in the entity profile database. The entity can initiate generating a nutrient composition by the processing module, wherein no further entity information may be provided by the entity when an entity profile with sufficient entity data exists for that entity in the entity profile database. The threshold for sufficient entity data can be set by the processing module. In particular, the processing module may differentiate core entity data and auxiliary entity data. The core entity data is required to generate a nutrient composition and the auxiliary entity data may supplement the core entity data to achieve greater accuracy of the nutrient composition, but may not be necessarily available. Accuracy of the nutrient composition may be defined as the delta between the objectively optimal nutrient composition and the nutrient composition generated by the processing module. More specifically, the nutrients included and their dosage may impact the accuracy of the nutrient composition.

The processing module can compose a nutrient composition based on the received external entity data and/or entity data provided by the node and/or nutrient data provided by a system database. However, the processing module may also compose a nutrient composition based on entity data stored in the entity profile database. This can be advantageous when the node does provide entity data or the entity data provided by the node is insufficient to generate a nutrient composition.

Furthermore, the system may comprise a receiver node which can receive a nutrient composition by the processing module. This may separate the input, respectively initiation of generating a nutrient composition, for example by a node and the receiving of a nutrient composition, in particular by the receiver node. The node may accept input from the entity, wherein the receiver node may only provide the nutrient composition as an output. The receiver node may, for example, be located at a pharmacy, medical facility, nutrition store, warehouse or integrated into an online store system. Moreover, the node can authenticate the receiver node to the processing module for receiving the nutrient composition composed by the processing module. The nutrient composition to be received by the receiver node may be based on the entity data provided by the respective node which authenticates the receiving of the nutrient composition by the receiver node.

Additionally, the system may comprise a data aggregation module configured to gather product nutrient(s) data from a product nutrient database. In particular the data aggregation module may access multiple product nutrient databases and/or manage requests by the processing module to access a certain product nutrient database.

The system according may further comprise the product nutrient database which is configured to store the product nutrient(s) data. The product nutrient data can comprise relational data. The product nutrient database may be a relational database linking several nutrient products and providing information on the nutrients comprised in each nutrient product.

The product nutrient(s) data stored in the product nutrient database can relate to food compositions. For example, the nutrient composition generated by the processing module can comprise food products, in particular based on their nutrient composition. Moreover, the processing module may generate a nutrient composition from a product nutrient database comprising selected meals and/or food related ingredients.

The processing module can generate a product nutrient composition on the basis of an updated template nutrient composition, wherein the product nutrient composition includes at least one nutrient with a nutrient property different from that nutrient property of the at least one nutrient in the product nutrient(s) datasets available in the product nutrient(s) database. A nutrient property can, for example, be the dosage, the delivery form, the bioavailability, maximum thresholds, minimum threshold and/or generally any quantity impacting the health of the individual. For example, using the template nutrient composition, i.e. the dosage or delivery form of the at least one nutrient can be altered in the generated nutrient composition.

The data aggregation module can gather product nutrient data from the product nutrient database(s) and manage storing of the product nutrient data in the system database. Management of the product nutrient data may comprise reading, writing, structuring and/or updating database entries. In particular the data aggregation module may group, rank, sort and/or link nutrient products based on the nutrient(s) comprised by the respective nutrient products. Data entries treated in such a way may be stored in the original product nutrient database and/or stored in a separate product nutrient database module.

Furthermore, the data aggregation module can calibrate nutrient data from the product nutrient data. The feature of calibrating nutrient data can, in particular, be defined as assigning efficacy values with regard to each contained nutrient, assigning bioavailability, standardizing values across products and/or normalize values across products for the respective nutrient products. This pertains, for example, to the homogenization of units, measurement procedures and/or labelling standards.

According to an advantageous embodiment of the invention calibrating nutrient data can refer to adjusting the bioavailability value for multiple nutrient products to allow a cross product comparison of bioavailability regarding the specific nutrients contained in the respective products.

According to a further advantageous embodiment the data aggregation module can be integrated with the processing module. Thus, the access to entity data and product nutrient(s) data can be centralized in one module.

Additionally, the aggregation module can generate relational data between a plurality of product nutrient data entries, wherein each product nutrient data entry relates to a specific nutrient product. The product nutrient data entry can be stored in the product nutrient(s) database. Relational data can, for example, be defined as combinatorial data between products, recommended combinations and/or combined use, wherein combined use as an attribute describes whether the nutrients, respectively the nutrient products can be consumed together, at the same time of day and/or have the same consistency. A plurality of nutrient products can be linked via the relational data. The relational data can indicate whether combining the respective products in a nutrient composition is favorable or unfavorable or which measures and limitations apply in combining the respective nutrient products. The data aggregation module can include the relational data in the nutrient data stored in the system database.

The processing module can compose a nutrient composition based on the relational data or at least taking the relational data into account. In particular, the processing module can generate a plurality of nutrient compositions, each including a different combination of nutrient products, and weigh the plurality of nutrient compositions according to the relational data to determine the most efficient, respectively the most suitable nutrient composition. The determined nutrient composition may then be transmitted to the node as the final output.

The processing module can control at least one of:
a. accessing the system database;
b. communication with a node;
c. processing a query from the node;
d. composing an individualized nutrient composition based on entity data;
e. transmitting an individualized nutrient composition to the node.

Accessing the system database can pertain to sending a request to retrieve product nutrient data and receiving product nutrient data from the system database. Alternatively, the processing module can access the system database via the aggregation module as a proxy and/or access the product nutrient(s) database to retrieve the desired data. Communication with the node can comprise initializing a communication session with the node and/or retrieving entity data from the node, in particular via the entity node interface as a proxy. The entity data can be received from the node sequentially, i.e. interrupted by interim requests from the processing module, and/or in bulk, meaning as a single data transfer from the node to the processing module. A query from the node can, for example, be a request to compose nutrient composition, advance dialog, initiate submission of entity data and/or confirm the submission of entity data.

The processing module can repeatedly capture product nutrient data and produce nutrient data from the product nutrient data. In particular, the product nutrient data can be captured continuously, event based, trigger based and/or periodically scheduled. Thus, the product nutrient data available for generating a nutrient composition to the processing module can be kept up to date to reflect possible changes in the product nutrient(s) data. The processing module can capture the product nutrient data from the system database, via the data aggregation module from the product nutrient(s) database and/or directly from the product nutrient(s) database.

The data aggregation module can receive a trigger signal and pull or receive product nutrient data from the product nutrient database and produce updated nutrient data in the system database according to the product nutrient data upon receiving the trigger signal. To generate a trigger signal the system can comprise a database update trigger module which can send a trigger signal to the data aggregation module to initiate an update of the nutrient data in the system database. In particular, the trigger signal can be generated when a change of available product nutrient data, for example, in the product nutrient database is detected. Thus, the system database can be kept up to date and a mismatch between the system database and available product nutrients data can be avoided or at least reduced.

The processing module can compose a template nutrient composition which is based on at least one entity data variable. The entity data variable represents an entity attribute, wherein a plurality of entities can have the same attribute or the same attribute range. The entity attribute can be, for example, age, gender, body mass index, blood pressure, heart rate (average, peak), level of physical exercise or any other variable suitable to categorize entities. Furthermore, the processing module can compose a template nutrient composition on the basis of at least one entity dataset. This at least one entity dataset can be provided by the node.

The template nutrient composition based on the at least one entity data can serve as a starting point for generating a nutrient composition regarding a further set of entity data, wherein the at least one entity dataset and the further set of entity data match in at least one entity variable. A template can serve as a blueprint for generating future nutrient compositions, in particular, with reduced entity data input to the processing module and/or reduced database requests and/or processing power by the processing module.

The processing unit can generate a template by statistically processing a plurality of entity datasets. The template can be optimized regarding preferences of the entity specific to the nutrient products, in particular to increase a customer lifetime value of that entity regarding a specific nutrient products supplier. The processing module can generate the template nutrient composition from a history of generated nutrient compositions specific to at least one entity.

Furthermore, the processing module can anonymize the template nutrient composition. Thereby, the advantage can be achieved, that the entity dataset remains identifiable but is unlinked from the particular entity that provided the entity profile. Thereby, possible infringement of data protection rights of the entity can be avoided or reduced in chance when statistically processing multiple entity datasets to generate a template nutrient composition.

The system can further comprise a template database and the processing module can store the template nutrient composition in the template database. Thus, the processing module can choose a suitable template nutrient composition according to the entity data provided. In particular, the processing module can match the provided entity data to a template nutrient composition by comparing and/or correlating the provided entity data to the entity data on which the respective template nutrient composition is based. When a respective comparison or correlation threshold is achieved the processing module can output the template nutrient composition to the node which provided the submitted entity data, in particular instead of generating a new individual nutrient composition.

Additionally, the processing module can update the template nutrient composition based on receiving second entity data, wherein the first entity data and the second entity data match qualitatively in at least one entity datum, present in the first entity data and the second entity data. Thus, the advantage is achieved that a template nutrient composition can be refined regarding that at least one entity datum. In particular, when limited entity data is available a template nutrient composition which is based on entity data that shares similar entity data values with the current entity dataset can be used to substitute generating a nutrient composition or serve as a basis to generate a nutrient composition by the processing module.

The processing module can compose a nutrient composition on the basis of the template nutrient composition and second entity data. In particular, the second entity data can be provided by the external database, entity profile database and/or a node. Thus, the advantage can be achieved, that the node requesting a nutrient composition does not need to provide entity data itself. The node can grant the processing module access to the second entity data.

According to a further advantageous embodiment a first node can provide the first entity data and a second node can provide the second entity data. In particular, the first entity data and the second entity data can supplement each other to form a complete entropy dataset on which the processing module can base the generating of the nutrient composition. Furthermore, the first entity data and the second entity data can comprise a specific entity data element wherein the value of that specific entity data element is different varies from the first entity set to the second entity set. The processing module can thus treat the respective specific entity data element with a respective uncertainty margin, disregard the specific entity data element when generating the nutrient composition, and/or request additional entity data from the first node or from the second node before generating the nutrient composition.

The node can provide feedback information to the processing module directly and/or via the node interface module. The feedback information can be input manually by an entity into the node or generated automatically by the node. The feedback information can pertain to a mismatch or partial mismatch of the generated nutrient composition with regard to the requirements of the entity. The requirements of the entity may not be directly represented by single entity data elements, but may be represented indirectly by a combination of single entity elements. Thus, the feedback information, in particular a statistical evaluation of multiple entries of feedback information, allows the processing module to recognize links between entity data elements and to increase the precision when generating nutrient compositions.

Additionally, the processing module can process the feedback information to increase the precision of a subsequently generated nutrient composition regarding nutrient specific factors, adjust a nutrient product shipping frequency and/or adjust a dosage form of nutrients by altering the type nutrient products included in the nutrient composition. A nutrient specific factor can be the total concentration of a nutrient included in a plurality of products of the nutrient composition. Additionally, the processing module can alter the dosage and/or delivery form of the respective nutrient. This can in particular be achieved by altering the nutrient product combination or the relative dosage of nutrient products with respect to each other.

The processing module can generate nutrient dosage information for at least one nutrient included in the nutrient composition when generating the nutrient composition. The nutrient dosage information can be part of the nutrient composition. The nutrient dosage information can be entity specific. The dosage information can include information on the spread of a single nutrient across multiple nutrient products to achieve a combined nutrient dosage. The dosage information can specify an intake value pertaining to the amount of nutrient to be consumed or absorbed by the entity per unit time.

The processing module can generate product dosage information for at least one nutrient product included in the nutrient composition when generating the nutrient composition. The processing module can generate a nutrient composition aiming at a homogenous dosage of the included nutrient products, i.e. a specific dose per predetermined time interval. The predetermined time interval preferably is an hour, a day, a week, a fortnight, a month or a year. Furthermore, the product dosage information can pertain to the combination of nutrient products. In particular, the processing unit can homogenize the product dosage information, i.e. a set number of units per nutrient product per specified time interval. A set number of units may, for example, be a volume of liquid, a number of granular nutrient compounds, i.e. pills and/or a set weight of a nutrient compound. Homogenization of the nutrient dosage information can pertain to providing identical or similar dosage information for at least two nutrient products included in the nutrient composition. Homogenization can be achieved by a combination of sustained release formulation or combining products with varying release time factors for the sustained release to achieve homogenized dosage information with different nutrient dosages. The nutrient dosage information can be linked to a product dosage information to create comprehensible dosage instructions which detail the overall nutrient dosage information and how that dose is split between the nutrient products included in the nutrient composition.

According to a preferred embodiment the processing module can generate a plurality of product dosage information on the basis of at least one nutrient dosage information. Additionally, the processing module can generate the at least one dosage information as part of the nutrient composition. Thus, the possibility of a mal dosage by the entity can be minimized. The product dosage information can contain use instructions regarding a nutrient product, i.e. when the nutrient product requires manual processing steps before consumption. Furthermore, the processing module can generate auxiliary information which indicates which entity data affected the generated nutrient dosage and provide the auxiliary information with the nutrient composition. This enables the entity receiving the nutrient composition to manually trace the nutrient composition. This can achieve the advantage of building trust by the entity to the automatic generation of nutrient combinations by processing module. The auxiliary information may further include warnings, i.e. listing adverse effects, when a dosage deviating from the given dosage information may be consumed. Furthermore, the auxiliary information can include information and/or data regarding the effects of nutrients included in the nutrient composition. In particular, when a nutrient may not have exclusively positive effects, but for the provided entity data the given dosage may outweigh possible disadvantages. Accordingly, the processing module can generate a risk index regarding a nutrient comprised in the nutrient composition to rate its overall effect on the entity.

The processing module can generate time validity data when generating the nutrient composition. The time validity data may pertain to a suggested time of consumption for a nutrient product included in the nutrient composition, i.e. consuming the suggested nutrient may be limited to a specific time of day, day of the week or more generally a recurring timing. Furthermore, the time validity data can pertain to the consumption of a nutrient regarding seasons or other weather or climate related events. In particular, the consumption of a nutrient can be linked to temperature, humidity, UV-index, precipitation, air quality and/or pollen concentration. Thus, the nutrient composition can be individualized regarding the environment of the entity. Additionally, the processing module can estimate the rate of change of provided entity data elements based on the generated nutrient composition including the dosage information pertaining to the included nutrient products and thus set a time validity limit for the provided nutrient composition in order to avoid mal dosing, in particular overdosing, of the nutrients comprised in the nutrient composition. Once the time limit included in the time validity data is reached the entity may need to provide updated entity data to receive an updated nutrient composition. The first generated nutrient composition can therefore include update interval information, specifying when the entity should provide updated entity data. Furthermore, the processing module can generate volume information and include the volume information in the generated nutrient composition. The volume information can pertain to the required nutrient product volume or nutrient product weight required to sufficiently provide the entity with nutrients according to the provided dosage information and/or time validity data.

Further, the processing module can generate a plurality of nutrient compositions and assign each nutrient composition specific time validity data. This can in particular relate to nutrient compositions which include nutrients that may only be required for a limited time period and/or nutrient compositions that are to be consumed sequentially, i.e. alternately or one after the other.

The processing module can generate time varying dosage information for a nutrient included in the generated nutrient composition. Thereby, a nutrient gradient can be created, in particular, to adjust the entity to the presence and/or consumption of a specific nutrient. For example, the dosage can be increased on a daily, weekly or monthly basis. Furthermore, this can be combined with time validity data so as to slowly decrease a nutrient concentration to the end of a time validity data of the respective nutrient composition.

According to a further advantageous embodiment, the processing module can compose a plurality of equivalent nutrient compositions on the basis of the entity data. The equivalent nutrient compositions comprise similar nutrient concentrations and differ in at least one nutrient product. The plurality of nutrient compositions may differ in overall price, availability, shipping time, manufacturer composition and/or nutrient product volume. A nutrient composition from the plurality of nutrient compositions which is to be provided to the entity can be selected by a master node, i.e. a manufacturer or nutrient product supplier. Alternatively, the plurality of nutrient compositions can be provided to the entity and/or node, which can then choose one of the nutrient compositions from the plurality of nutrient compositions. Accordingly, the processing module can transmit the plurality of nutrient compositions to the node.

The processing module can select a preferred nutrient composition from the plurality of nutrient compositions on the basis of product specific data. The product specific data can pertain to technical aspects of the nutrient products, i.e. product size, nutrient delivery form, included volume, weight, included number of single doses. Furthermore, the product specific data can pertain to non-technical aspects of the nutrient products, i.e. estimated delivery time, nutrient product price, overall nutrient composition price, availability of the complete, respectively partial, nutrient composition and/or manufacturer preference to sell, available stock. Specifically, the preferred nutrient composition can be aimed at providing a nutrient composition in a cost and time efficient manner for the nutrient composition provider, i.e. a reseller or manufacturer, as well as for the entity. Preferably, with regard to the above embodiment, the processing module can transmit the preferred nutrient composition to the node.

Furthermore, the processing module can gather product specific data from the system database. The availability of product specific data by the system database can limit the dependence of the processing module on external data sources and in particular conflicting formats or conflicting nutrient product data when generating the nutrient composition.

The processing module can conform the entity data to a data norm. A data norm can be a defined data format, scale, unit system and/or laboratory data format including value thresholds. In particular, the processing module can scale and/or convert measurement values, i.e. laboratory values to a system uniform scale and/or unit system. This can achieve the advantage of reducing scaling errors and subsequently providing erroneous nutrient compositions, for example, when required nutrient concentrations do not match the actual nutrient content provided with the nutrient composition.

According to a further advantageous embodiment the processing module can conform a bioavailability to a bioavailability norm. Bioavailability can be defined as the fraction of absorbed and utilized micronutrients, which can be particularly important for nonheme iron and provitamin A carotenoids as bioavailability can vary depending on a number of factors. Thus, it is important to integrate bioavailability into the composition of nutrient compositions to combat nutrient deficiencies, in particular iron and vitamin A deficiencies. More generally, bioavailability is the potential for uptake of a substance by an entity, i.e. a living organism. Thus, the bioavailability can define the amount of a nutrient, respectively an active component of a nutrient, which is available unaltered in the systemic cycle of the entity.

The node can include preference data in the entity data. The preference data can relate to the delivery form of the nutrient, dosage of the nutrient, time interval for which the nutrient composition shall cover the nutrient needs of the entity. Furthermore, the preference data can include nutrient depot strategies, wherein long-term release dosages of nutrients can be included to bridge periods in which no nutrients from the nutrient composition are to be consumed by the entity. Generally, the entity can provide individualized preferences on how to and when the nutrient products, respectively nutrients comprised in the nutrient composition can be consumed. Furthermore, the preference data can relate to future changes in the entity data, i.e. planned increased or decreased physical activity, changes in general nutrition. More generally this information can be subsumed as a health profile. Thus, the processing module can generate a nutrient composition catering to the health profile of an individual entity and the related estimated trajectory for required nutrients.

According to a further advantageous embodiment the processing module can comprises a plurality of data processing units, each being able to process the entity data using a set of weighting factors and/or significance thresholds. Each set is unique to the respective processing unit. For example, the data processing units can be distinguished by the type of nutrient composition each data processing unit can produce. The data processing units can, for example, be labeled as basic, intermediate and advanced with corresponding weighting factors.

The weighting factors of the basic processing unit can for example emphasize essential entity data and have reduced values for less essential entity data or omit the less essential entity data all together. In particular, the access to external entity can generate costs per retrieval of the external entity data, thus, the basic processing unit can generate a nutrient composition more cost efficient. Furthermore, the processing unit may generate the nutrient composition faster, as a request for external entity data can be omitted and also the available product nutrient(s) data can be limited to a subset.

The intermediate data processing unit can in turn use a larger set of higher weighting factors compared to the basic processing unit and in turn consider a wider range of entity data parameters. Consequentially, the intermediate data processing unit can generate a nutrient composition which includes basic nutrients and additional supplemental nutrients.

The advanced data processing unit can, compared to the basic or intermediate data processing unit, implement a reduced weighting, thus considering each entity data element to its full capacity. In particular, the advanced data processing unit can cross-link entity data to generate meta entity data of dependent or interacting entity data elements. For example, entity data specifying a high physical exercise activity and also specifying the location of the entity may indicate a higher demand for a specific nutrient. The advanced data processing unit can derive a required nutrient dosage by estimating altitude, likely elevation gain, type of terrain, air quality etc. Thus, the advanced data processing unit can generate meta entity data and base a sophisticated nutrient composition on the entity data and/or the meta entity data. In particular the advanced data processing unit can comprise increased thresholds for number of products, number of contained nutrients and/or overall price point of the nutrient composition. The advanced data processing unit can achieve the advantage of a higher estimated health benefit to the entity.

The processing module can select a data processing unit according to the preference data and thus can select a predefined mode for composing a nutrient composition based on the preference data. Furthermore, the entity, node, node interface module or master node can provide the respective preference data to select a data processing unit. In particular, preference data by the master node can supersede preference data submitted by the node, thus the master node can limit, respectively control access of the node to specific data processing units and in turn specific types of nutrient compositions.

According to an advantageous embodiment the node can comprise an input terminal configured to receive the entity data in form of a user input. The input terminal can be configured to receive manual, vocal, electronic or visual input and create an entity dataset from the received input. Electronic input can comprise an electronic message, respectively data packet, data obtained via device pairing, i.e. smartphone, medical device, health tracking device. Visual input can be provided in the form of a scan, respectively photo of a document.

According to a further advantageous embodiment the system can comprise master node which can provide a composing template to the processing module. The processing module can then compose nutrients according to the composing template. The composing template may include preference data regarding nutrient products, i.e. defining which nutrient product shall be included in the nutrient composition when equivalent choices exist and/or a choice based on entity data shall be superseded. Furthermore, the composing template can provide information on a base set of nutrient products to be included in the nutrient composition.

Furthermore, the processing module can select a subset of entries of the system database on the basis of the composing template and to compose nutrients on the basis of the selected subset of entries of the system database. Thus, information provided in form of the composing template can, for example, limit the available nutrient products on which the nutrient composition is based. Thus, nutrient compositions which are based on a specific product range can be generated.

The master node can be one of the nodes.

The processing module can perform a multidimensional optimization when generating the nutrient composition. Thus, the processing module can perform a multiple criteria decision making optimizing for more than one objective simultaneously. This is in particular relevant, where an optimal nutrient composition can only be achieved with trade-offs between two or more conflicting objectives. For example, minimizing cost while maximizing the efficacy of provided nutrients. Generally, no single solution exists that simultaneously optimizes each objective. Thus, determining which nutrient composition to generate, respectively choose, depends on additional preference information.

Performing the multidimensional optimization can include at least two variables to be optimized. These can for example be a set of two from the following: cost to manufacture, margin, expected customer lifetime value, fulfilling estimated nutrient requirement, cost to the entity, bioavailability of the included nutrients.

On the basis of the multidimensional optimization the processing module can generate an optimized combination of nutrients. The processing module can match existing nutrient products from the product nutrient(s) database to the optimized combination of nutrients to generate a nutrient composition.

According to a further advantageous embodiment of the invention the processing module can generate a nutrient composition(s) to be formalized in nutrient product(s) on the basis of the optimized combination of nutrients. Thus, the optimized combination of nutrients can serve as a basis to create a new nutrient product to be included in the product nutrient(s) database. In other words, on the basis of the gathered entity data an optimized combination of nutrients can be generated which can serve as a blueprint to develop new nutrient products.

The invention is further described with the following numbered embodiments.

Below, system embodiments will be discussed. These embodiments are abbreviated by the letter “S” followed by a number. Whenever reference is herein made to “system embodiments”, these embodiments are meant.
S1. A system for automatically composing individualized nutrient compositions from a plurality of nutrient(s) comprising products, the system comprising a processing module (1) configured to receive entity data and to individually compose nutrients on the basis of the entity data.
S2. System according to the preceding system embodiment, wherein the processing module (1) is configured to receive and process product nutrient(s) data and/or to generate a nutrient composition based on the entity data and nutrient(s) data.
S3. System according to any one of the preceding system embodiments further comprising a node (20-22), wherein the node (20-22) is configured to provide the entity data.
S4. System according to the preceding system embodiment, wherein the node (20-22) is configured to receive the entity data in form of a user input.
S5. System according to any of the preceding system embodiments with the features of S3, wherein the node (20-22) is configured to communicate with the processing module (1).
S6. System according to any of the preceding system embodiments with the features of S3, wherein the node (20-22) is configured to authenticate an entity to the processing module (1).
S7. System according to any one of the preceding system embodiments with the features of S3, further comprising a node interface module (4) being configured to communicate with the node (20-22) and to receive the entity data.
S8. System according to the preceding system embodiment, wherein the node interface module (4) is configured to receive local product nutrient(s) data from a medical and/or pharmaceutical database module, wherein the local product nutrient(s) data comprises nutrient product data locally available at the node interface module (4).
S9. System according to the preceding system embodiment, wherein the node interface module (4) is configured to provide the local product nutrient(s) data to the processing module (1) which is configured to compose a nutrient composition based on the local product nutrient(s) data.
S10. System according any of to the preceding system embodiments with features of S7, wherein the node (20-22) is configured to authenticate an entity to the processing module (1) and/or the node interface module (4).
S11. System according to any of the preceding system embodiments with the features of S6 and S7, wherein the node interface module (4) and or the processing module (1) is/are configured to authenticate the entity, the user and/or the node (20-22) by means of authentication provided by the node (20-22).
S12. System according to the preceding system embodiment with the features of S7, wherein the processing module (1) and/or the node interface module (4) is/are configured to receive the entity data when the node (20-22) is authenticated.
S13. System according to any of the preceding system embodiments with the features of S7, wherein the node interface module (4) is integrated with the processing module (1).
S14. System according to any of the preceding system embodiment with the features of S7, wherein the node interface module (4) is configured to trigger receipt of the entity data by the processing module (1) and/or the node interface module (4).
S15. System according to any of the preceding system embodiments with the features of S7, wherein the node is configured to trigger transmission of the entity data to the node (20-22) interface module (4) and/or the processing module (1).
S16. System according to any of the preceding system embodiments with the features of S7, wherein the node interface module (4) is configured to communicate with an external database module (13) to receive entity data.
S17. System according any of the preceding system embodiment with the features of S3 and S7, wherein the node (20-22) is configured to authenticate a communication of the node interface module (4) with the external database module (13) to initiate a transmission of entity data from the external database module (13) to the node interface module (4), wherein the transmitted entity data is specific to the entity represented by the node which authenticates the communication.
S18. System according to any of the 2 preceding system embodiments, wherein the node interface module (4) is configured to authenticate the node (20-22) and/or itself to the external database module (13) to receive entity data specific to the entity represented by the node (20-22).
S19. System according to any of the preceding system embodiments, wherein the processing module (1) is configured to communicate with the external database module (13) to receive entity data.
S20. System according to the preceding system embodiments with the features of S3, wherein the node (20-22) is configured to authenticate a communication of the processing module (1) with the external database module (13) to initiate a transmission of entity data from the external database module (13) to the processing module (1), wherein the transmitted entity data is specific to the entity represented by the node (20-22) which authenticates the communication.
S21. System according to any of the 2 preceding system embodiments, wherein the processing module (1) is configured to authenticate the node (20-22) and/or itself to the external database module (13) to receive entity data specific to the entity represented by the node (20-22).
S22. System according to any of the preceding system embodiments further comprising the external database module (13).
S23. System according to the preceding system embodiment, wherein the external database module (13) is configured to provide entity data to the processing module (1).
S24. System according to system embodiment S22 with features of S7, wherein the external database module (13) is configured to provide entity data to the node interface module (4).
S25. System according to system embodiment S23 or S24, wherein the entity data provided by the external database module (13) is specific to one individual and pertains to the health condition, nutrient consumption, pharmaceutical intake, physical exercise and/or medical data of that individual.
S26. System according to any one of the preceding system embodiments with features of S3 and S7 wherein the node (20-22) is configured to activate the node interface module (4) and wherein the node interface module (4) is configured to be activated by the node (20-22) and configured to trigger communication with one or more of the node(s) (20-22) upon activation.
S27. System according to the preceding system embodiment, wherein the node(s) (20-22) is/are configured to trigger communication with the entity interface module (4) upon activation.
S28. System according to any of the preceding system embodiments with the features of S7, wherein the node interface module is configured to communicate in dialogue with the entity node(s).
S29. System according to any of the preceding system embodiments with the features of S7, further comprising an entity profile database (3) for storing entity data.
S30. System according to the preceding system embodiment with the features of S3, wherein the processing module (1) is configured to store entity data provided by the nodes (20-22) in the entity profile database (3).
S31. System according to any of the 2 preceding system embodiments, wherein the processing module (1) is configured to store entity data provided by the external database module (13) in the entity profile database (3).
S32. System according to any of the 2 preceding system embodiments with the features of S22, wherein the processing module (1) is configured to merge first entity data provided by the nodes (20-22) and second entity data provided by the external database module (13) to generate merged entity data, wherein the processing module (1) is configured to compose nutrients on the basis of the merged entity data.
S33. System according to the preceding system embodiment wherein the processing module (1) and/or the user interface module (4) is/are configured to anonymize the received entity data and wherein the entity profile database (3) is configured to store the anonymized entity data.
S34. System according to any preceding system embodiments, wherein the processing module (1) is configured to receive external entity data from the external database module and to compose a nutrient composition based on the received external entity data.
S35. System according to the preceding system embodiment with the features of S2, wherein the processing module (1) is configured to filter and/or select product nutrient(s) data on which generating the nutrient composition is based.
S36. System according to the preceding system embodiment, wherein the processing module (1) is configured to select a subset of product nutrient data to minimize a product interaction potential and/or a pharmaceutical interaction potential and/or an allergic reaction potential for the entity when the generated nutrient composition is consumed.
S37. System according to any preceding system embodiments comprising an external database module (13) configured to store entity data provided by an external source.
S38. System according any of to the preceding system embodiments with features of S29 wherein the node interface module (4) is configured to receive first entity data provided by the node (20-22), to communicate with the external database module (13) to receive second entity data, to integrate the first entity and second entity data to generate combined entity data and to store the combined entity data in the entity profile database (3).
S39. System according to the preceding system embodiment, wherein the processing module (1) is configured to compose a nutrient composition based on the received external entity data and/or entity data provided by the node (20-22) and/or nutrient data provided by a system database (2).
S40. System according to any of the 3 preceding system embodiments, wherein the processing module (1) is configured to compose a nutrient composition based on entity data stored in the entity profile database (3).
S41. System according to any of the preceding system embodiments, comprising a receiver node (15) wherein the receiver node (15) is configured to receive a nutrient composition by the processing module.
S42. System according to the preceding system embodiment with the features of S3, wherein the node (20-22) is configured to authenticate the receiver node (15) to the processing module (1) for receiving of the nutrient composition composed by the processing module (1).
S43. System according to any one of the preceding system embodiments further comprising a data aggregation module (9) configured to gather product nutrient(s) data from a product nutrient database (10-12).
S44. System according to any one of the preceding system embodiments further comprising the product nutrient database (10-12) which is configured to store the product nutrient(s) data.
S45. System according to the preceding system embodiment, wherein the product nutrient(s) data stored in the product nutrient(s) database (10-12) relates to food compositions.
S46. System according to any of the preceding system embodiments with features of S43 and S60, wherein the processing module (1) is configured to generate a product nutrient composition on the basis of the updated template nutrient composition, wherein the product nutrient composition includes at least one nutrient with a nutrient property different from that nutrient property of the at least one nutrient in the product nutrient(s) datasets available in the product nutrient(s) database.
S47. System according to any of the preceding system embodiments with features of S43 comprising the system database (2) wherein the data aggregation module (9) is configured to gather product nutrient data from the product nutrient database(s) (10-12) and to manage storing of the product nutrient data in the system database (2).
S48. System according to any of the preceding system embodiments with features of S43, wherein the data aggregation module (9) is configured to calibrate nutrient data from the product nutrient data.
S49. System according to any of the preceding system embodiments with features of S43, wherein the data aggregation module (9) is integrated with the processing module (1).
S50. System according to any of the preceding system embodiments with features of S43, wherein the data aggregation module (9) is configured to generate relational data between a plurality of product nutrient data entries, wherein each product nutrient data entry relates to a specific nutrient product.
S51. System according to the preceding system embodiment, wherein the data aggregation module (9) is configured to include the relational data in the nutrient data stored in the system database (2).
S52. The system according to any of the preceding system embodiments, wherein the processing module (1) is configured to compose a nutrient composition based on the relational data.
S53. The system according to any of the preceding system embodiments with the features of S3 and S47 wherein the processing module (1) is configured to control at least one of:
   a. accessing the system database (2);
   b. communication with a node (20-22);
   c. processing a query from the node (20-22);
   d. composing an individualized nutrient composition based on entity data;
   e. transmitting an individualized nutrient composition to the node (20-22).
S54. The system according to any of the preceding system embodiments wherein the processing module (1) is configured to repeatedly capture product nutrient data and produce nutrient data from the product nutrient data.
S55. The system according to any of the preceding system embodiments with features of S43 and S47 wherein the data aggregation module (9) is configured to receive a trigger signal and to pull or receive product nutrient data from the product nutrient database (10-12) and produce updated nutrient data in the system database (2) according to the product nutrient data upon receiving the trigger signal.
S56. The system according to the preceding system embodiment comprising a database update trigger module (16), which is configured to send a trigger signal to the data aggregation module (9) to initiate an update of the nutrient data in the system database (2).
S57. The system according to any of the preceding system embodiment with the features of S3, wherein the processing module (1) is configured to compose a template nutrient composition, wherein the template nutrient composition is based on at least one entity data variable.
S58. The system according to the preceding system embodiment, wherein the processing module (1) is configured to anonymize the template nutrient composition.
S59. The system according to any of the 2 preceding system embodiments, comprising a template database (17), wherein the processing module (1) is configured to store the template nutrient composition in the template database (17).
S60. The system according to any of the 3 preceding system embodiments, wherein the processing module (1) is configured to update the template nutrient composition based on further entity data, wherein the further entity data matches qualitatively in at least one entity datum, on which the template nutrient composition is based.
S61. The system according to any of the 4 preceding system embodiments, wherein the processing module (1) is configured to compose a nutrient composition on the basis of the template nutrient composition and second entity data.
S62. The system according to the preceding system embodiment with the features of S3, wherein a first node (20) of the nodes (20-22) is configured to provide the first entity data and wherein a second node (21) of the nodes (20-22) is configured to provide the second entity data.
S63. The system according to any of the preceding system embodiments with features of S3 and S7, wherein the node (20-22) is configured to provide feedback information to the processing module (1) directly and/or via the node interface module (4).
S64. The system according to the preceding system embodiment, wherein the processing module (1) is configured to process the feedback information to increase the precision of a subsequently generated nutrient composition regarding nutrient specific factors, adjust a nutrient product shipping frequency and/or adjust a dosage form of nutrient products.
S65. The system according to any of the preceding system embodiments, wherein the processing module (1) is configured to generate nutrient dosage information for at least one nutrient included in the nutrient composition when generating the nutrient composition.
S66. The system according to any of the preceding system embodiments, wherein the processing module (1) is configured to generate a plurality of product dosage information on the basis of at least one nutrient dosage information, wherein the processing module (1) is configured to generate the at least one dosage information as part of the nutrient composition.
S67. The system according to any of the preceding system embodiments, wherein the processing module (1) is configured to generate time validity data when generating the nutrient composition.
S68. The system according to any of the preceding system embodiments, wherein the processing module (1) is configured to generate a plurality of nutrient compositions and assign each nutrient composition specific time validity data.
S69. The system according to any of the preceding system embodiments, wherein the processing module (1) is configured to generate time varying dosage information for a nutrient included in the generated nutrient composition.
S70. The system according to any of the preceding system embodiments, wherein the processing module (1) is configured to compose a plurality of equivalent nutrient compositions on the basis of the entity data, wherein the equivalent nutrient compositions comprise similar nutrient concentrations and differ in at least one nutrient product.
S71. The system according to the preceding system embodiment with the features of S3, wherein the processing module (1) is configured to transmit the plurality of nutrient compositions to the node (20-22).
S72. The system according to any of the preceding system embodiments with the features of S70, wherein the processing module (1) is configured to select a preferred nutrient composition from the plurality of nutrient compositions on the basis of product specific data.
S73. The system according to the preceding system embodiment with the features of S47 wherein the processing module (1) is configured to gather product specific data from the system database (2).
S74. The system according to any of the 2 preceding system embodiments with the features of S3, wherein the processing module is configured to transmit the preferred nutrient composition to the node (20-22).
S75. The system according to any of the preceding system embodiments wherein the processing module (1) is configured to conform the entity data to a data norm.
S76. The system according to any of the preceding system embodiments wherein the processing module (1) is configured to conform a bioavailability to a bioavailability norm.
S77. The system according to any of the preceding system embodiments with the features of S3, wherein the node (20-22) is configured to include preference data in the entity data.
S78. The system according any of to the preceding system embodiments wherein the processing module (1) comprises a plurality of data processing units (1.1-1.3), wherein each data processing unit (1.1-1.3) is configured to process the entity data using a set of weighting factors and/or significance thresholds, wherein each set is unique to the respective processing unit (1.1-1.3).
S79. The system according to the preceding system embodiment with features of S77, wherein the processing module is configured to select a data processing unit (1.1-1.3) according to the preference data.
S80. The system according to any of the preceding system embodiments with the features of S3, wherein the node (20-22) comprises an input terminal configured to receive the entity data in form of a user input.
S81. The system according to any of the preceding system embodiments comprising a master node (23) configured to provide a composing template to the processing module (1) and wherein the processing module (1) is configured to compose nutrients according to the composing template.
S82. The system according to the preceding system embodiment with the features of S47 wherein the processing module (1) is configured to select a subset of entries of the system database (2) on the basis of the composing template and to compose nutrients on the basis of the selected subset of entries of the system database (2).
S83. The system according to any of the 2 preceding system embodiments with the features of S3 wherein the master node (23) is one of the nodes (20-22).
S84. The system according to any of the preceding system embodiments, wherein the processing module (1) is configured to perform a multidimensional optimization when generating the nutrient composition.
S85. The system according to the preceding system embodiment, wherein performing the multidimensional optimization includes at least two variables to be optimized.
S86. The system according to any of the preceding system embodiments wherein the system is configured to carry out the method according to any of the method embodiments.

Below, method embodiments will be discussed. These embodiments are abbreviated by the letter “M” followed by a number. Whenever reference is herein made to “method embodiments”, these embodiments are meant.
M1. A method for automatically composing individualized nutrient compositions from a plurality of nutrient(s) comprising products, the method comprising receiving of entity data and individually composing nutrients on the basis of the entity data by a processing module (1)
M2. The method according to the preceding method embodiment, comprising receiving and processing product nutrient(s) data and/or generating a nutrient composition based on the entity data and nutrient(s) data.
M3. The method according to any one of the preceding method embodiments comprising providing the entity data by a node (20-22).
M4. The method according to the preceding method embodiment comprising receiving the entity data in form of a user input by the node (20-22).
M5. The method according to any one of the preceding method embodiments comprising communicating between the processing module (1) and the node (20-22).
M6. The method according to any one of the preceding method embodiments comprising authenticating an entity to the processing module (1) by the node (20-22).
M7. The method according to any one of the preceding method embodiments comprising communicating between the node (20-22) and a node interface module (4) for receiving the entity data from the node (20-22) by the node interface module (4).
M8. The method according to any one of the preceding method embodiments comprising receiving local product nutrient(s) data from a medical and/or pharmaceutical database module by the node interface module (4), wherein the local product nutrient(s) data comprises nutrient product data locally available at the node interface module (4).
M9. The method according to the preceding method embodiment comprising providing the local product nutrient(s) data to the processing module (1) by the node interface module (4) and composing a nutrient composition based on the local product nutrient(s) data by the processing module (4).
M10. The method according to any one of the preceding method embodiments comprising authenticating an entity to the processing module (1) and/or the node interface module (4) by the node (20-22).
M11. The method according to any one of the preceding method embodiments comprising authenticating the entity, the user and/or the node (20-22) by the processing module (1) and/or the node interface module (4) using means of authentication provided by the node (20-22).
M12. The method according to any one of the preceding method embodiments comprising receiving the entity data when the node (20-22) is authenticated by the processing module (1) and/or the node interface module (4).
M13. The method according to any one of the preceding method embodiments comprising triggering receipt of the entity data by the processing module (1) and/or the node interface module (4).
M14. The method according to any one of the preceding method embodiments comprising triggering transmission of the entity data to the node interface module (4) and/or the processing module (1) by the node (20-22).
M15. The method according to any one of the preceding method embodiments comprising communicating between an external database module (13) and the node interface module (4) to receive entity data at the node interface module (4).
M16. The method according to any one of the preceding method embodiments comprising authenticating a communication of the node interface module (4) with the external database module (13) to initiate a transmission of entity data from the external database module (13) to the node interface module (4) by the node (20-22), wherein the transmitted entity data is specific to the entity represented by the node authenticating the communication.
M17. The method according to any one of the preceding method embodiments comprising authenticating the node (20-22) and/or the node interface module (4) to the external database module (13) for receiving entity data specific to the entity represented by the node (20-22) by the node interface module (4).
M18. The method according to any one of the preceding method embodiments comprising communicating of the processing module (1) with the external database module (13) to receive entity data.
M19. The method according to any one of the preceding method embodiments comprising authenticating a communication of the processing module (1) with the external database module (13) to initiate transmitting of entity data from the external database module (13) to the processing module (1) by the node (20-22), wherein the transmitted entity data is specific to the entity represented by the node (20-22) authenticating the communication.
M20. The method according to any one of the preceding method embodiments comprising authenticating the node (20-22) and/or the processing module (1) to the external database module (13) by the processing module (1) to receive entity data specific to the entity represented by the node (20-22).
M21. The method according to any one of the preceding method embodiments comprising providing entity data to the processing module (1) by the external database module (13).
M22. The method according to any one of the preceding method embodiments comprising provide entity data to the node interface module (4) by the external database module (13).
M23. The method according to any one of the preceding method embodiments comprising activating the node interface module (4) by the node (20-22) and triggering a communication between the node interface module (4) with one or more of the node(s) (20-22) upon activation.
M24. The method according to any one of the preceding method embodiments comprising communicating in dialogue with the entity node(s) (20-22) by the node interface module (4).
M25. The method according to any one of the preceding method embodiments comprising storing entity data provided by the nodes (20-22) in an entity profile database (3) by the processing module (1).
M26. The method according to any one of the preceding method embodiments comprising storing entity data provided by the external database module (13) in the entity profile database (3) by the processing module (4).
M27. The method according to any one of the preceding method embodiments comprising merging first entity data provided by the nodes (20-22) and second entity data provided by the external database module (13) to generate merged entity data and composing nutrients on the basis of the merged entity data by the processing module (1).
M28. The method according to any one of the preceding method embodiments comprising anonymizing the received entity data by the processing module (1) and/or the user interface module (4) and storing the anonymized entity data by the entity profile database (3).
M29. The method according to any one of the preceding method embodiments comprising receiving external entity data from the external database module and to composing a nutrient composition based on the received external entity data by the processing module (1).
M30. The method according to any one of the preceding method embodiments comprising filtering and/or selecting product nutrient(s) data on which generating the nutrient composition is based by the processing module (1).
M31. The method according to any one of the preceding method embodiments comprising selecting a subset of product nutrient(s) data by the processing module (1) to minimize a product interaction potential and/or a pharmaceutical interaction potential and/or an allergic reaction potential for the entity when the generated nutrient composition is consumed.
M32. The method according to any one of the preceding method embodiments comprising storing entity data provided by an external source by the external database module (13).
M33. The method according to any one of the preceding method embodiments comprising receiving first entity data provided by the node (20-22), communicating with the external database module (13) to receive second entity data, integrating the first entity and second entity data to generate combined entity data and storing the combined entity data in the entity profile database (3) by the node interface module (4).
M34. The method according to any one of the preceding method embodiments comprising composing a nutrient composition by the processing module (1) based on the received external entity data and/or entity data provided by the node (20-22) and/or nutrient data provided by a system database (2).
M35. The method according to any one of the preceding method embodiments comprising composing a nutrient composition based on entity data stored in the entity profile database (3) by the processing module (1).
M36. The method according to any one of the preceding method embodiments comprising receiving a nutrient composition generated by the processing module at a receiver node (15).
M37. The method according to any one of the preceding method embodiments comprising authenticating the receiver node (15) to the processing module (1) by the node (20-22) for receiving of the nutrient composition composed by the processing module (1).
M38. The method according to any one of the preceding method embodiments comprising gathering product nutrient(s) data from a product nutrient database (10-12) by a data aggregation module (9).
M39. The method according to any one of the preceding method embodiments comprising storing the product nutrient(s) data by the product nutrient database (10-12).
M40. The method according to any one of the preceding method embodiments with the steps of M53 comprising generating a product nutrient composition by the processing module (1) on the basis of the updated template nutrient composition, wherein the product nutrient composition includes at least one nutrient with a nutrient property different from that nutrient property of the at least one nutrient in the product nutrient(s) datasets available in the product nutrient(s) database.
M41. The method according to any one of the preceding method embodiments comprising gathering product nutrient data from the product nutrient database(s) (10-12) and managing storing of the product nutrient data in the system database (2) by the data aggregation module (9).
M42. The method according to any one of the preceding method embodiments comprising calibrating nutrient data from the product nutrient data by the data aggregation module (9).
M43. The method according to any one of the preceding method embodiments comprising generating relational data between a plurality of product nutrient data entries by the data aggregation module (9), wherein each product nutrient data entry relates to a specific nutrient product.
M44. The method according to any one of the preceding method embodiments comprising including the relational data in the nutrient data stored in the system database (2) by the data aggregation module (9).
M45. The method according to any one of the preceding method embodiments comprising composing a nutrient composition based on the relational data by the processing module (1).
M46. The method according to any one of the preceding method embodiments comprising
   a. accessing the system database (2);
   b. communicating with a node (20-22);
   c. processing a query from the node (20-22);
   d. composing an individualized nutrient composition based on entity data;
   e. transmitting an individualized nutrient composition to the node (20-22), wherein the steps a. to e. are controlled by the processing module (1).
M47. The method according to any one of the preceding method embodiments comprising capturing product nutrient data and producing nutrient data from the product nutrient data repeatedly by the processing module (1).
M48. The method according to any one of the preceding method embodiments comprising receiving a trigger signal and pulling, respectively receiving product nutrient data from the product nutrient database (10-12) by the data aggregation module (9) and producing updated nutrient data in the system database (2) according to the product nutrient data upon receiving the trigger signal by the data aggregation module (9).
M49. The method according to any one of the preceding method embodiments comprising sending a trigger signal to the data aggregation module (9) to initiate an update of the nutrient data in the system database (2) by a database update trigger module (16).
M50. The method according to any one of the preceding method embodiments comprising composing a template nutrient composition by the processing module (1) on the basis of first entity data provided by the node (20-22).
M51. The method according to the preceding method embodiment comprising anonymizing the template nutrient composition by the processing module (1).
M52. The method according to any one of the preceding method embodiments with the steps of M50 comprising storing the template nutrient composition in the template database (17) by the processing module (1).
M53. The method according to any one of the preceding method embodiments with steps of M50 comprising updating the template nutrient composition by the processing module (1) based on receiving second entity data, wherein the first entity data and the second entity data match qualitatively in at least one entity datum, present in the first entity data and the second entity data.
M54. The method according to the preceding method embodiment comprising with steps of M50 comprising composing a nutrient composition by the processing module (1) on the basis of the template nutrient composition and second entity data.
M55. The method according to any one of the preceding method embodiments comprising providing the first entity data by first node (20) of the nodes (20-22) and providing the second entity data by a second node (21) of the nodes (20-22).
M56. The method according to any one of the preceding method embodiments comprising providing feedback information to the processing module (1) directly and/or via the node interface module (4) by the node (20-22).
M57. The method according to any one of the preceding method embodiments with steps of M56 comprising processing the feedback information by the processing module (1) to increase the precision of a subsequently generated nutrient composition regarding nutrient specific factors, adjusting a nutrient product shipping frequency and/or adjusting a dosage form of nutrient products by the processing module (1).
M58. The method according to any one of the preceding method embodiments comprising generating nutrient dosage information for at least one nutrient included in the nutrient composition the processing module (1) when generating the nutrient composition.
M59. The method according to any one of the preceding method embodiments comprising generating a plurality of product dosage information on the basis of at least one nutrient dosage information by the processing module (1), and generating the at least one dosage information as part of the nutrient composition by the processing module (1).
M60. The method according to any one of the preceding method embodiments comprising generating time validity data when generating the nutrient composition by the processing module (1).
M61. The method according to any one of the preceding method embodiments comprising generating a plurality of nutrient compositions and assigning each nutrient composition specific time validity data by the processing module (1).
M62. The method according to any one of the preceding method embodiments comprising generating time varying dosage information for a nutrient included in the generated nutrient composition by the processing module (1).
M63. The method according to any one of the preceding method embodiments comprising composing a plurality of equivalent nutrient compositions on the basis of the entity data by the processing module (1), wherein the equivalent nutrient compositions comprise similar nutrient concentrations and differ in at least one nutrient product.
M64. The method according to any one of the preceding method embodiments comprising with the steps of M63, transmitting the plurality of nutrient compositions to the node (20-22) by the processing module (1).
M65. The method according to any one of the preceding method embodiments with the steps of M63 comprising selecting a preferred nutrient composition from the plurality of nutrient compositions on the basis of product specific data by the processing module (1).
M66. The method according to any one of the preceding method embodiments comprising gathering product specific data from the system database (2) by the processing module (1).
M67. The method according to any one of the preceding method embodiments with steps of M65 comprising transmitting the preferred nutrient composition to the node (20-22) by the processing module (1).
M68. The method according to any one of the preceding method embodiments comprising conforming the entity data to a data norm by the processing module (1).
M69. The method according to any one of the preceding method embodiments comprising conforming a bioavailability to a bioavailability norm by the processing module (1).
M70. The method according to any one of the preceding method embodiments comprising including preference data in the entity data by the node (20-22).
M71. The method according to any one of the preceding method embodiments comprising processing the entity data using a set of weighting factors and/or significance thresholds by data processing units (1.1-1.3), wherein each set is unique to the respective processing unit (1.1-1.3).
M72. The method according to any one of the preceding method embodiments with steps of M70 comprising selecting a data processing unit (1.1-1.3) according to the preference data by the processing module (1).
M73. The method according to any one of the preceding method embodiments comprising receiving the entity data in form of a user input by an input terminal of the node (20-22).
M74. The method according to any one of the preceding method embodiments comprising providing a composing template to the processing module (1) by a master node (23) and composing nutrients according to the composing template by the processing module (1).
M75. The method according to any one of the preceding method embodiments comprising selecting a subset of entries of the system database (2) on the basis of the composing template and composing nutrients on the basis of the selected subset of entries of the system database (2) by the processing module (1).
M76. The method according to any one of the preceding method embodiments comprising performing a multidimensional optimization by the processing module (1) when generating the nutrient composition.
M77. The method according to any of the preceding method embodiments wherein the method is a computer implemented method.

### BRIEF DESCRIPTION OF FIGURES

The present invention will now be described with reference to the accompanying drawings, which illustrate embodiments of the invention. These embodiments should only exemplify, but not limit, the present invention.

FIG. 1 schematically depicts an embodiment of the present invention;

FIG. 2 schematically depicts a further embodiment of the present invention;

FIG. 3 schematically depicts a further embodiment of the present invention;

FIG. 4 schematically depicts a further embodiment of the present invention;

FIG. 5 schematically depicts a further embodiment of the present invention;

FIG. 6 schematically depicts a further embodiment of the present invention;

FIG. 7 schematically depicts a further embodiment of the present invention;

FIG. 8 schematically depicts a further embodiment of the present invention;

FIG. 9 schematically depicts a further embodiment of the present invention;

FIG. 10 schematically depicts a further embodiment of the present invention;

### DETAILED DESCRIPTION OF FIGURES

It is noted that not all the drawings carry all the reference signs. Instead, in some of the drawings, some of the reference signs have been omitted for sake of brevity and simplicity of illustration. Embodiments of the present invention will now be described with reference to the accompanying drawings.

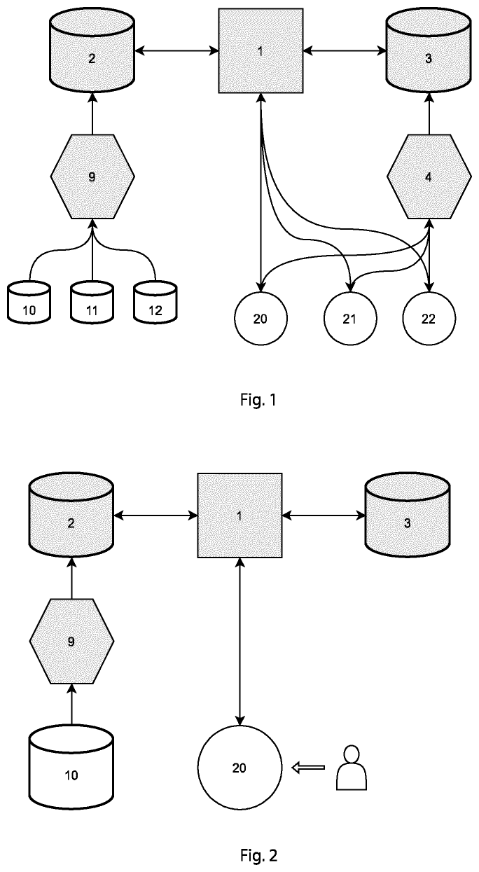

FIG. 1 schematically depicts an embodiment of a system and a respective method to generate a personalized data-based nutritional composition in accordance with the present invention.

As is apparent, that there are a number of product nutrient data sources or databases 10-12 with data of different origin available. As has been mentioned, the data contained in those product nutrient databases 10-12 can represent data with nutrition information or data of different suppliers, different territories, different kinds of food or drinks etc. In an embodiment in accordance with the present invention, each product nutrient database 10-12 can represent data of one supplier and their respective nutrient-containing products. The present invention can be applied for one supplier offering different nutrients, i.e. different products comprising a single nutrient or a plurality of nutrients. Different products can contain the same nutrient in a different form or concentration. The nutrient form can relate to the chemical variant of the nutrient or its dosage respectively delivery form.

In a next step, the data in the different databases 10-12 can be preprocessed in a respective nutrient database interface 9. The nutrient database interface 9 can approach the different databases 10-12 and can store the respective content in a system database 2, which may be separate or integrated with the product nutrient databases 10-12). The product nutrient databases 10-12 can either trigger the approaching by the nutrient database interface 9 or the nutrient database interface 9 can contact the product nutrient databases 10-12 on its own motion and/or regularly etc. The respective communication can then renew any information in the system database 2.

The data to each supplier can be handled separately although it is stored in the system database 2. This system database 2 can be easily accessed by the processing component 1.

Alternatively, the processing component can be connected or connect itself to the different databases directly. According to an embodiment of the invention the processing module 1 can alternatively be connected with each different product nutrient database 10-12 via a dedicated communication channel and a dedicated processing component 1 can be also assigned or implemented to each different product nutrient database 10-12.

The data in system database 2 can be calibrated in order to be able to better compare the data regarding the nutrients.

In FIG. 1 are also a number of nodes 20-22 depicted. The nodes 20-22 can be accessed by users or end customers or can be accessed by other networks, such as doctor's networks.

In any case, a node interface module 4 can be provided that is configured to centrally manage node data provided by at least one of the nodes 20-22 transmitted by the nodes 20-22. In particular, the node data can comprise biometric data, medical data, personal information and more generally any entity specific data. This node data can be personalized in a manner fulfilling privacy and/or data protection requirements but nevertheless making it possible for an individual to access his/her personal data and to have their personal data considered when a nutrient composition is generated.

An entity profile database 3 can collectively store the entity data. This entity profile database 3 can be approached by the processing module 1. However, the processing module 1 may also communicate with the nodes 20-22 directly.

The entity data can comprise data relating to the user or individual, such as gender, age, peculiarities such as allergies etc. Also, genetic or epi-genetic data or any other data may be assigned to the individual. For that reason, also other databases can be brought into communication with the entity profile database and/or the node interface module can be configured to complement entity data provided by a node 20-22 by accessing an external database.

In FIG. 1 components of one or more modules in line with the present invention are highlighted with a grey shade.

Any nodes 20-22 can approach the processing module 1 in order to obtain individualized and wholistic data for an individual profile.

The managing component 1 can be configured to also enable communication, encryption, safe transmittal of data etc.

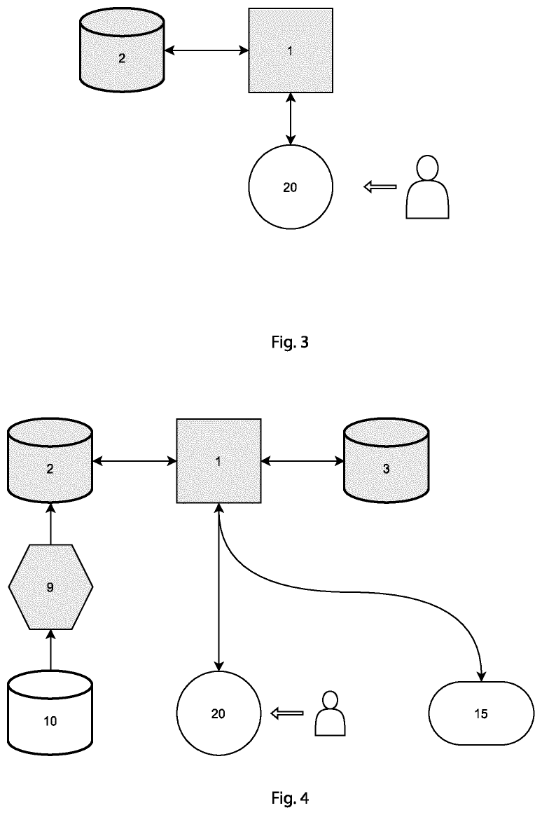

FIG. 2 schematically depicts an embodiment of a system and a respective method to generate a personalized data-based nutritional composition in accordance with the present invention. An entity, in particular a user, can provide entity data pertaining to that user to the node 20. Overall, the system embodiment depicted in FIG. 2 can be considered as a generalization of the embodiment shown in FIG. 1. The communication between the node 20 and the processing module 1 can include a node interface module 4 as a proxy. However, the node 20 and the processing module can communicate directly to transfer entity data to the processing module 1 and to transfer a generated nutrient composition from the processing module 1 to the node 20.

Bar a failed authorization by the node, the entity data provided by the node 20 can be stored in the entity profile database 3 for future reference by the processing module 1. In particular, the user may grant the processing module access to previously stored entity data via the node 20. Furthermore, the user can provide updated entity data via the node 20 to be stored in the entity profile database 3, in particular to update an existing entity data entry specific to said user.

The data aggregation module 9 can be fed with product nutrient(s) data from a single product nutrients database 10 or a plurality of databases 10-12. In particular, the data aggregation module 9 can convert the received product nutrient(s) data to a format accessible by the processing module 1 and store the converted product nutrient(s) data in the system database 2. Access to the product nutrient(s) database 10 by the data aggregation module 9 can be restricted to be read only, thereby preserving the integrity of the product nutrient(s) database 10. The data contained in the system database 2 may be altered by the data aggregation module 9 and/or the processing module 1. According to an embodiment of the invention the user may provide product nutrient(s) data via the node 20 and then via the processing module 1. Furthermore, the user can provide product nutrient(s) data just to the processing module 1 for use in generating a requested nutrient composition.

In particular, the user can provide data on nutrient products at hand, i.e. by scanning the packaging of the nutrient product or by inserting information manually. Thus, the processing module 1 can include nutrient products already available to the user in the nutrient composition output to the node respectively the user.

FIG. 3 schematically depicts an embodiment of a system and a respective method to generate a personalized data-based nutritional composition in accordance with the present invention. Overall, the system embodiment depicted in FIG. 3 can be considered as a generalization of the embodiment shown in FIG. 2. In particular, the processing module can operate based on initially provided product nutrient(s) data stored in the system database 2. A user can provide entity data directly to the processing module 1 via the node 20. This embodiment can be a minimalistic, in particular a self-contained, stand-alone version, where no external database input is needed to generate a nutrient composition.

The content of the system database 2 can be periodically and/or manually updated to include recent product nutrient(s) data. The entity data provided by the node 20 can be provided for each nutrient composition to be generated.

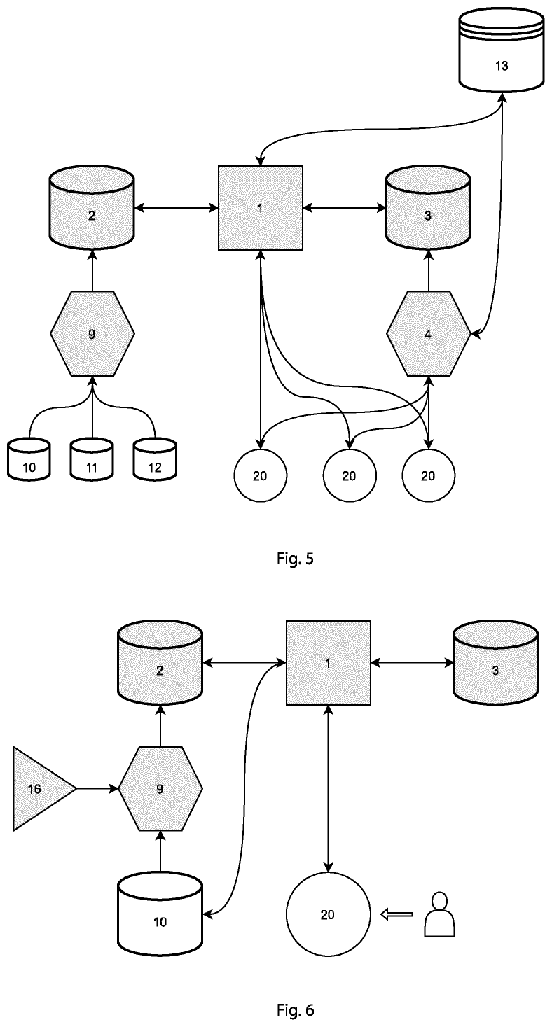

FIG. 4 schematically depicts an embodiment of a system and a respective method to generate a personalized data-based nutritional composition in accordance with the present invention.

According to an embodiment of the invention the system comprises a receiver node 15 configured to communicate with the processing module 1. In particular, the receiver node can be configured to receive a nutrient composition composed by the processing module 1. Thus, input in the form of entity data provided by the user can be separated from the output in form of the generated nutrient composition. The receiver node 15 can be configured to initiate a shipping, respectively handing over of at least one nutrient product included in the nutrient composition. The receiver node 15 can be a part of a shipping system, product handling system or a retail system. The receiver node 15 can be inaccessible to manipulation and/or input from the entity providing the entity data to the node 20.

Additionally, the receiver node 15 can be configured to handle a payment process with the node 20 or via the processing module as a proxy. Alternatively, the user may interact with the receiver node to issue a payment to receive nutrient products included in the nutrient composition sent to the receiver node 15 by the processing module 1.

The receiver node can be supervised by a medical and/or pharmaceutical entity. The processing module can be configured to include prescription only nutrient products in the nutrient composition. Such nutrient compositions may be limited to be sent to a receiver node and may be inaccessible for fulfillment at the node. The receiver node can be configured to handle the transfer of prescription only products to the entity, respectively the user. To include prescription only products, the system database can contain entries relating to prescription only products to be included in a nutrient composition.

FIG. 5 schematically depicts an embodiment of a system and a respective method to generate a personalized data-based nutritional composition in accordance with the present invention. The embodiment depicted in FIG. 5 can be viewed as a variant of the embodiment shown in FIG. 1 with the system comprising an external database module 13.

The node interface module 4 can communicate with the external database module 13 to receive entity data and/or to transmit entity data. This provides the advantage that additional entity data, in particular entity data not available from the node or not transmitted by the node, can be gathered and used as a basis for generating the nutrient composition. Alternatively, one of the nodes 20-22 can send updated entity data to the node interface module 4 which in turn can update the entity data of that respective entity stored in the external database module 13. The above-mentioned functions can also be performed by the processing module 1.

For that purpose, the node can authenticate the communication of the processing module 1 and/or the node interface module 4 with the external database module. The transmitted entity data can be specific to the entity represented by the respective node which authenticates the communication. Thus, the entity can have full control over the specific entity data which is provided to the node interface module 4 and/or the processing module 1. In particular, the entity can enforce limits regarding which entity data is available to the node interface module 4, respectively the processing module 1 from the external database 13.

The node interface module 4 can authenticate each of the nodes 20-22 and/or itself to the external database module 13 to receive entity data specific to the entity represented by respective node 20-22. The external database module 13 may provide access to medical data regarding the entity. Thus, it is advantageous to limit access to the external database module 13 to authenticated entities, respectively an authenticated node 20-22 and/or node interface module 4. The authentication can include a verification of the entity and authorization of access to a data transfer from the external database module to the node interface module 4 and/or the processing module 1. Therefore, the processing module 1 can be configured to communicate with the external database module 13.

Each node 20-22 can authenticate a communication of the processing module 1 with the external database module 13 to initiate a transmission of entity data specific to the entity represented by the respective node 20-22 from the external database module 13 to the processing module 1. The transmitted entity data is specific to the entity represented by the respective node 20-22 which authenticates the communication. This achieves the advantage that the entity data received from the external database module 4 can be directly sent to the processing module 1. In particular, the nodes 20-22 can grant access to the processing module 1 and/or the node interface module 4 to gather additional entity data from the external database module 13. Thus, the amount of entity data provided by the respective node 20-22 can be reduced to data which is not yet present in the system, especially not present in the external database module 13 or an entity profile database linked to the processing module 1. The entity profile database can be a template database.

The processing unit 1 can authenticate the nodes 20-22 and/or itself to the external database module 13 to receive entity data specific to the entity represented by the respective node 20-22. Thereby, achieving the advantage that the nodes 20-22 do not need to provide authentication information. The nodes 20-22, respectively the entities providing entity data through the node can authenticate themselves to the processing module 1 while the processing module 1 manages the authentication to the external database module 13.

The external database module 13 can provide entity data to the node interface module 4 and/or to the processing module 1. The entity data provided by the external database module 13 can be specific to one individual, respectively one entity and pertains to the health condition, nutrient consumption, pharmaceutical intake, physical exercise and/or medical data of that individual. Furthermore, the entity data provided by the external database module 4 can comprise nutritional-, exercise-, geolocation- and/or body function-tracking data. This data can be supplied by devices handled by, respectively linked to the entity. This achieves the advantage that the processing module 1 is able to generate a nutrient composition based on detailed information concerning the health state of the individual, respectively entity. Thereby, greater accuracy of the provided nutrient composition. In particular, the external database module 13 can initiate a communication with a personal health data tracker wherein a request to receive entity data from that personal health data tracker can be authorized by the entity, user or individual via one of the nodes 20-22.

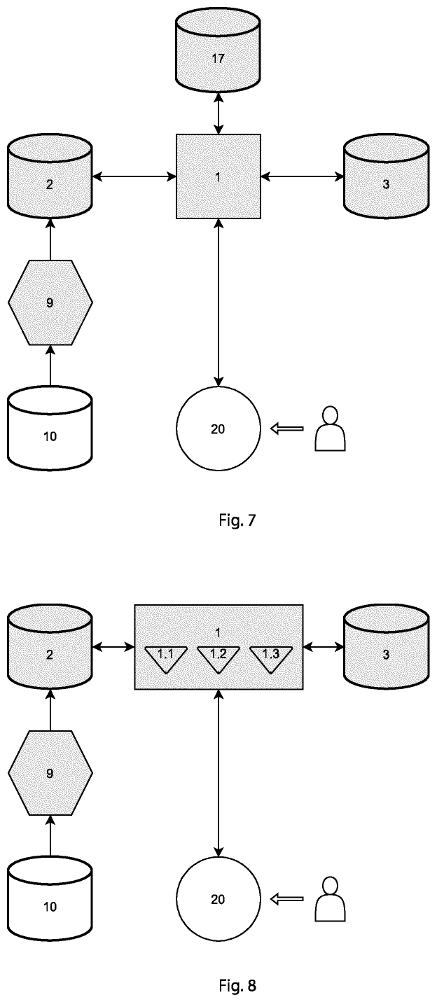

FIG. 6 schematically depicts an embodiment of a system and a respective method to generate a personalized data-based nutritional composition in accordance with the present invention. The embodiment depicted in FIG. 6 can be viewed as a variant of the embodiment shown in FIG. 2 with the system comprising a database update trigger module 16.

The processing module 1 and/or the nutrient database module 9 can repeatedly capture product nutrient(s) data and produce nutrient data from the product nutrient data, wherein the nutrient data can be stored in the system database 2. In particular, the product nutrient(s) data can be captured trigger based. Thus, the product nutrient data available for generating a nutrient composition to the processing module 1 can be kept up to date to reflect possible changes in the product nutrient(s) data. The processing module 1 can capture the product nutrient data from the system database 2, via the data aggregation module 9 from the product nutrient(s) database and/or directly from the product nutrient(s) database 10.

The data aggregation module 9 can receive a trigger signal from the database update trigger module 16 and pull or receive product nutrient(s) data from the product nutrient database 10 and produce updated nutrient data in the system database 2 according to the product nutrient data gathered upon receiving the trigger signal. The database update trigger module 16 can send a trigger signal to the data aggregation module 9 to initiate an update of the nutrient data in the system database 2. In particular, the trigger signal can be generated when a change of available product nutrient data, for example, in the product nutrient database 10 is detected. Thus, the system database 2 can be kept up to date and a mismatch between the system database 2 and available product nutrients data can be avoided or at least reduced.

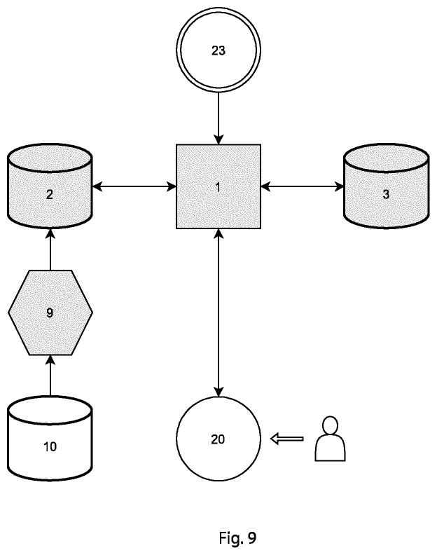

FIG. 7 schematically depicts an embodiment of a system and a respective method to generate a personalized data-based nutritional composition in accordance with the present invention. The embodiment depicted in FIG. 7 can be viewed as a variant of the embodiment shown in FIG. 2 with the system comprising a template database 17.

The processing module 1 can generate and store a template nutrient composition in the template database 17. The processing module 1 can choose a suitable template nutrient composition which correlates to the entity data provided. In particular, the processing module 1 can match the provided entity data to a template nutrient composition by comparing and/or correlating the provided entity data to the entity data on which the respective template nutrient composition is based. More specifically, the processing module 1 can compare entity data elements and check if they are identical or estimate a delta to the respective entity data element linked to the template nutrient composition.

When a respective comparison or correlation threshold is achieved the processing module 1 can output the template nutrient composition to the node 20 which provided the submitted entity data. Thus, the processing module 1 can provide the template nutrient composition parallel to or instead of generating a new nutrient composition.

Additionally, the processing module 1 can update the template nutrient composition based on receiving second entity data, wherein the first entity data and the second entity data match qualitatively in at least one entity datum, present in the first entity data and the second entity data. Thus, the advantage is achieved that a template nutrient composition can be refined regarding that at least one entity datum.

The processing module 1 can compose a nutrient composition on the basis of the template nutrient composition and second entity data. Thereby, the generated nutrient composition can be understood as an incremental refinement of the base template nutrient composition. The second entity data can be provided by the external database, entity profile database and/or the node 20. The node 20 can grant the processing module 1 access to the second entity data.

FIG. 8 schematically depicts an embodiment of a system and a respective method to generate a personalized data-based nutritional composition in accordance with the present invention. The embodiment depicted in FIG. 8 can be viewed as a variant of the embodiment shown in FIG. 2 with the system comprising a plurality of data processing units 1.1-1.3.

Each data processing unit 1.1-1.3 can process entity data using a set of weighting factors and/or significance thresholds. Each set of weighting factors and/or significance thresholds is unique to the respective processing unit 1.1-1.3. For example, the data processing units 1.1-1.3 can be distinguished by the type of nutrient composition each data processing unit can produce based on the different factors. The data processing unit 1.1 can be a basic unit, the data processing unit 1.2 can be an intermediate unit and/or the data processing unit 1.3 can be an advanced unit.

The weighting factors of the basic processing unit 1.1 can, for example, emphasize essential entity data, thus a first set of high weighting factors can be applied to the essential entity data and/or a second set of low weighting factors can be applied to non-essential entity data. The set of high weighting factors can comprise weighting factors higher than the weighting factors comprised in the set of low weighting factors. The processing module and/or the respective processing unit 1.1-1.3 can be configured to select a set of essential entity data and a set of non-essential entity data from the submitted entity data. The selected sets of entity data can be subsets of the submitted entity data which, when combined, constitute the full set of provided entity data. The intersection of the selected subsets can contain no entity data elements.

The access to external entity can generate costs per retrieval of the external entity data, thus, the basic processing unit 1.1 can generate a nutrient composition more efficiently with regard cost and processing time and generate a nutrient composition solely on the basis of the entity data provided by the node 20. The decrease in processing time can be achieved since the basic processing unit 1.1 can omit a request for external entity data and/or limit the available product nutrient(s) data to a smaller subset. Thus, communication with other modules and

The intermediate data processing unit 1.2 can in turn use a larger set of high weighting factors compared to the basic processing unit and in turn put emphasis on a wider range of entity data parameters. Consequentially, the intermediate data processing unit 1.2 can, for example, generate a nutrient composition which includes basic nutrients and additional supplemental nutrients. Moreover, the intermediate data processing unit 1.2 can generate a nutrient composition which can achieve a higher match between the objective nutrient needs of the entity and the nutrients provided by the nutrient composition, when compared to the nutrient composition generated by the basic processing unit 1.1.

The advanced data processing unit 1.3 can, compared to the basic or intermediate data processing units 1.1-1.2, implement a reduced weighting, thus considering each entity data element to its full capacity. In particular, the advanced data processing unit 1.3 can cross-link entity data to generate meta entity data of dependent or interacting entity data elements. Furthermore, the advanced data processing unit 1.3 can comprise increased thresholds for number of products comprised in the nutrient composition, number of contained nutrients in the nutrient products included in the nutrient composition and/or overall price point of the nutrient composition. The advanced data processing unit 1.3 can achieve the advantage of a higher estimated health benefit to the entity. In particular, when compared to the basic and/or intermediate processing unit 1.1-1.2 the advanced processing module 1.3 can generate a nutrient composition offering nutrients to the entity with the smallest delta to the objective nutrient requirement of the entity.

The processing module 1 can select a data processing unit 1.1-1.3 according to preference data submitted by a node 20. Moreover, the preference data can be made available to the processing module 1 by a master node, i.e. a manufacturer- and/or supplier-controlled node. Thus, the processing module 1 can select a predefined mode for composing a nutrient composition based on the preference data. Furthermore, the entity, node 20, node interface module or master node can provide the respective preference data to select a data processing unit 1.1-1.3. In particular, preference data by the master node can supersede preference data submitted by the node 20, thus the master node can limit, respectively control access of the node 20 to specific data processing units 1.1-1.3 and in turn specific types of nutrient compositions.

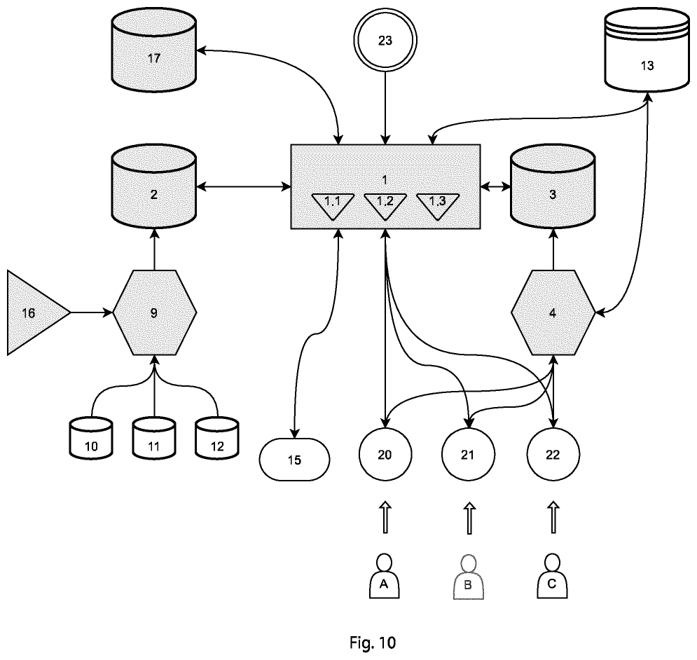

FIG. 9 schematically depicts an embodiment of a system and a respective method to generate a personalized data-based nutritional composition in accordance with the present invention. The embodiment depicted in FIG. 9 can be viewed as a variant of the embodiment shown in FIG. 2 with the system comprising a master node 23.

The master node 23 can be a manufacturer- and/or supplier-controlled node or a node controlled by a medical, a pharmaceutical and/or a pharmacological entity. Furthermore, the master node 23 can provide preference data to select a data processing unit and/or to define a subset of product nutrient(s) data on which generating a nutrient composition is to be based to the processing module 1. In particular, preference data by the master node 23 can supersede preference data submitted by the node 20. Thus, the master node 23 can limit, respectively control access of the node 20 to specific nutrient products and in turn specify the range of nutrient compositions available to the node 20.

According to an advantageous embodiment of the invention the master node 23 can provide a composing template to the processing module 1. The processing module 1 can then compose nutrients according to the composing template. The composing template may include preference data regarding nutrient products, i.e. defining which nutrient product shall be included in the nutrient composition when equivalent choices exist and/or a choice based on entity data shall be superseded. Furthermore, the composing template can provide information on a base set of nutrient products to be included in the nutrient composition.

FIG. 10 schematically depicts an embodiment of a system and a respective method to generate a personalized data-based nutritional composition in accordance with the present invention. The embodiment depicted in FIG. 10 can be viewed as a specialized variant of the embodiments shown in FIGS. 1 and 4 to 9. In particular, the system can receive entity data from three nodes 20, 21, 22 which represent the respective entities, i.e. users A, B, C.

The processing module 1 can select a data processing unit 1.1-1.3 according to preference data which can be made available to the processing module 1 by an entity A, B, C via the respective node 20-22 and/or by a manufacturer- and/or supplier-via a master node 23. The processing module 1 can select a predefined mode for composing a nutrient composition based on the preference data. Furthermore, the entity, node 20, node interface module 4 or master node 23 can provide the respective preference data to select a data processing unit 1.1-1.3. In particular, preference data by the master node 23 can supersede preference data submitted by the node 20, thus the master node 23 can limit, respectively control access of the node 20 to specific data processing units 1.1-1.3 and in turn specific types of nutrient compositions.

The system comprises a system database 2 for storing product nutrient(s) data. The system database 2 can be updated by receiving data from the processing module 1 and/or the product nutrient databases 10-12, in particular via the data aggregation module 9. The data aggregation module 9 can receive a trigger signal from the database update trigger module 16 and pull or receive product nutrient(s) data from the product nutrient databases 10-12 and produce updated nutrient data in the system database 2 according to the product nutrient data gathered upon receiving the trigger signal.

In conjunction with an update of data stored in the system database 2 the processing module can recalibrate the entries in the system database 2. Updating the system database can further include removing and/or adding product nutrient(s) data and/or edit existing database entries.

The database update trigger module 16 can send a trigger signal to the data aggregation module 9 to initiate an update of the nutrient data in the system database 2. In particular, the trigger signal can be generated when a change of available product nutrient data, for example, in the product nutrient database 10 is detected. Additionally, the trigger signal can be generated periodically to ensure updates and consistency between the system database and the product nutrient(s) databases 10-12. The data aggregation module 9 can check for duplicate entries found in the product nutrient databases 10-12 to include only a single entry for the respective product in the system database 2.

The system comprises an entity profile database 3 which can collectively store the entity data. This entity profile database 3 can be approached by the processing module 1. Furthermore, the processing module 1 may also communicate with the nodes 20-22 directly and/or via the node interface module 4.

Each user A, B, C can provide updated entity data via its respective node 20-22 to be stored in the entity profile database 3. An update to the entity data stored in the system database 2 can constitute replacing existing values with more recent values.

According to an advantageous embodiment of the present invention the updated entity data can be stored as a separate entry in the entity database 2 so as to generate an entity data history. In particular, the processing unit 1 can then be configured to base generating a nutrient composition for the respective entity on particular changes of the most recently supplied entity data to previously added entity datasets of the respective entity.

The external database module 13 can provide entity data to the node interface module 4 and/or to the processing module 1. The external database 13 can process a request transmitted, respectively authorized by a node 20-22 to provide entity data to the processing module 1 and/or the node interface module 4. The entity data stored in the external database 13 can pertain to the respective entity, wherein the entity data itself is generated by a third party. For example, the external database can comprise medical test results, prescriptions and/or other, in particular confidential entity data. The third party can be a medical entity, pharmaceutical entity, public health service entity or a provider of bio tracking function services, i.e. health tracking devices, personal body parameter tracking devices, activity tracking devices.

The system comprises a receiver node 15 which can receive a nutrient composition composed by the processing module 1. Input in the form of entity data provided by a user A, B, C, via the nodes 20-22 can be separated from the output in form of the generated nutrient composition. The processing module can provide entity data together with the nutrient composition to the receiver node 15. In particular, the processing module can provide entity data suitable to identify the specific entity whose entity data is the basis for the provided nutrient composition. For example, the processing module 1 can provide an address, billing information, a name, electronic contact details, phone number pertaining to the respective entity and/or node.

The system comprises a template database 17. The processing module 1 can generate and store a template nutrient composition in the template database 17. Furthermore, the processing module 1 can update the template nutrient composition stored in the template database 17 based on further entity data, wherein the further entity data matches qualitatively in at least one entity datum, on which the template nutrient composition is based. The processing module 1 can identify clusters of entities by their shared entity data values and assign template nutrient composition to each entity cluster and store the respective cluster specific template nutrient compositions in the template database 17.

Reference numbers and letters appearing between parentheses in the claims, identifying features described in the embodiments and illustrated in the accompanying drawings, are provided as an aid to the reader as an exemplification of the matter claimed. The inclusion of such reference numbers and letters is not to be interpreted as placing any limitations on the scope of the claims.

The term “at least one of a first option and a second option” is intended to mean the first option or the second option or the first option and the second option.

Whenever a relative term, such as “about”, “substantially” or “approximately” is used in this specification, such a term should also be construed to also include the exact term. That is, e.g., “substantially straight” should be construed to also include “(exactly) straight”.

Whenever steps were recited in the above or also in the appended claims, it should be noted that the order in which the steps are recited in this text may be accidental. That is, unless otherwise specified or unless clear to the skilled person, the order in which steps are recited may be accidental. That is, when the present document states, e.g., that a method comprises steps (A) and (B), this does not necessarily mean that step (A) precedes step (B), but it is also possible that step (A) is performed (at least partly) simultaneously with step (B) or that step (B) precedes step (A). Furthermore, when a step (X) is said to precede another step (Z), this does not imply that there is no step between steps (X) and (Z). That is, step (X) preceding step (Z) encompasses the situation that step (X) is performed directly before step (Z), but also the situation that (X) is performed before one or more steps (Y1), . . . , followed by step (Z). Corresponding considerations apply when terms like “after” or “before” are used.

1-15. (canceled)

16. A system for automatically composing individualized nutrient compositions from a plurality of nutrients comprising products, the system comprising:
a processing module configured to receive entity data, receive product nutrient(s) data and to individually compose nutrients on the basis of the entity data and the product nutrient(s) data, and
a node, wherein the node is configured to receive entity data in form of a user input and provide the entity data to the processing module.

17. The system according to claim 16, further comprising a node interface module configured to communicate with the node and to receive the entity data, wherein the node interface module is configured to communicate with an external database module to receive further entity data.

18. The system according to claim 17, wherein the node is further configured to authenticate a communication of the node interface module and/or the processing module with the external database module to initiate a transmission of entity data from the external database module to the node interface module and/or the processing module, and wherein the transmitted entity data is specific to the entity represented by the node which authenticates the communication.

19. The system according to claim 17, wherein the node is further configured to activate the entity interface module and wherein the user interface is configured to be activated by the node and configured to trigger communication with one or more of the nodes upon activation.

20. The system according to claim 17, further comprising an entity profile database configured to store entity data, wherein the processing module is further configured to store entity data provided by the nodes and/or the external database module in the entity profile database and further configured to generate a nutrient composition based on the entity data stored in the entity profile database.

21. The system according to claim 16, wherein the processing module is further configured to generate a filter based on the provided entity data to reduce a product interaction potential, a pharmaceutical interaction potential, and/or an allergic reaction potential for the entity, wherein the processing module is further configured to apply the filter to the product nutrient(s) data to generate filtered product nutrient(s) data, and to generate a nutrient composition on the basis of the filtered product nutrient(s) data.

22. The system according to claim 16, further comprising a system database and a data aggregation module, wherein the data aggregation module is configured to gather product nutrient data from product nutrient database(s) and to manage storing of the product nutrient data in the system database, and wherein the processing module is further configured to compose a nutrient composition based on received external entity data and/or entity data provided by the node and/or nutrient data provided by the system database.

23. The system according to claim 22, wherein the data aggregation module is further configured to calibrate nutrient data from the product nutrient(s) data and store the calibrated nutrient data in the system database.

24. The system according to claim 22, wherein the data aggregation module is further configured to generate relational data between a plurality of product nutrient data entries, wherein each product nutrient data entry relates to a specific nutrient product, the data aggregation module further being configured to integrate the relational data with the product nutrient(s) data stored in the system database and wherein the processing module is further configured to process the relational data when composing a nutrient composition.

25. The system according to claim 17, wherein the node interface module is further configured to receive local product nutrient(s) data from a local database module and to communicate the local product nutrient(s) data and/or the entity data available at the node interface module to the processing module which is further configured to compose a nutrient composition based on the local product nutrient(s) data, wherein the local product nutrient(s) data comprises nutrient product(s) data locally available at the node interface module.

26. The system according to claim 16, wherein the processing module is further configured to compose a template nutrient composition on the basis of at least one entity dataset.

27. The system according to claim 26, wherein the processing module is further configured to update the template nutrient composition based on receiving further entity data, wherein the at least one entity dataset and the further entity data match in at least one entity data variable.

28. The system according to claim 16, wherein the processing module is further configured to generate nutrient dosage information for at least one nutrient included in the nutrient composition when generating the nutrient composition.

29. The system according to claim 16, wherein the processing module is further configured to generate time varying dosage information for a nutrient included in the generated nutrient composition.

30. The system according to claim 16, wherein the processing module is further configured to compose a plurality of equivalent nutrient compositions on the basis of the entity data, and wherein the equivalent nutrient compositions comprise similar nutrient concentrations and differ in at least one nutrient product, and
wherein the processing module is further configured to select a preferred nutrient composition from the plurality of nutrient compositions on the basis of product specific data.

31. The system according to claim 16, further comprising a master node configured to provide a composing template to the processing module and wherein the processing module is further configured to generate a nutrient composition on the basis of to the composing template.

32. The system according to claim 16, wherein the processing module is configured to perform a multidimensional optimization when generating the nutrient composition.

33. The system according to claim 32, wherein the processing module is further configured to generate an optimized combination of nutrients based on the multidimensional optimization and wherein the processing module is further configured to match existing nutrient products from the product nutrient(s) data to the optimized combination of nutrients to generate a nutrient composition.

34. A method for automatically composing individualized nutrient compositions from a plurality of nutrient(s) comprising products, the method comprising:
receiving entity data and individually composing nutrients on the basis of the entity data by a processing module, and
receiving and processing product nutrient(s) data and/or generating a nutrient composition based on the entity data and nutrient(s) data.

35. The method according to claim 34, further comprising:
merging first entity data provided by nodes and second entity data provided by an external database module to generate merged entity data and composing nutrients on the basis of the merged entity data by the processing module.
